(19) United States
(12) Patent Application Publication
(10) Pub. No.: US 2025/0295431 A1
(43) Pub. Date: Sep. 25, 2025

(54) EXPANDABLE SURGICAL ACCESS PORT WITH ELONGATED WORKSPACE

(71) Applicant: Vycor Medical, Inc., Boca Raton, FL (US)

(72) Inventors: Michael Phelps, Malvern, PA (US); David Cantor, Rome (IT); Robert Schaefer, Riverside, CA (US)

(73) Assignee: Vycor Medical, Inc., Boca Raton, FL (US)

(21) Appl. No.: 19/227,653

(22) Filed: Jun. 4, 2025

Related U.S. Application Data
(63) Continuation-in-part of application No. 18/217,089, filed on Jun. 30, 2023.
(60) Provisional application No. 63/358,651, filed on Jul. 6, 2022.

(51) Int. Cl. A61B 17/34; A61B 17/00

(52) U.S. Cl. CPC A61B 17/3423; A61B 17/3415; A61B 17/3439; A61B 2017/00982; A61B 2017/3425

(57) ABSTRACT

Provided is an expandable surgical access port having an arm mounting body defining an opening surrounding a longitudinal axis, a number of primary arms arranged around the longitudinal axis, a number of secondary arms arranged around the longitudinal axis, an activation ring rotatably mounted to the arm mounting body to pivot the primary and secondary arms from a closed position to an opened position, and a an expandable membrane extending along the primary and secondary arms. When arms are in the opened position, the expandable membrane has an elongated membrane cross-section as viewed along the longitudinal axis.

This application is a continuation-in-part of U.S. application Ser. No. 18/217,089 filed on Jun. 30, 2023, which claims the benefit of U.S. Provisional Application No. 63/358,651 filed on Jul. 6, 2022, the entire contents of both of which are incorporated herein by reference.

### BACKGROUND

A variety of access ports have been proposed and used for accessing the delicate tissue of the brain. Accessing brain tissue, especially interior brain tissue that is not accessible at the surface, poses a variety of challenges. For example, it is desirable to minimize damage to the brain tissue by a traumatically forming a pathway for access. To this end, surgical access ports typically have a tapered distal end to gently spread the tissue as the access port is moved into position at the surgery site. Upon reaching the surgery site, the central portion of the access port is removed or the access port is expanded to form a pathway for surgical instruments.

Another challenge with brain access ports is that the brain tissue can be damaged simply by contact with the wall of the access port. Intracranial pressure causes the brain tissue local to the access port to compress, potentially leading to brain ischemia—i.e., a reduction in blood flow and oxygen that can potentially lead to death of the local brain tissue. Such damage was particularly likely in the past, when flat blade or spoon retractors were used to create a surgical access pathway. The change to access ports having a full circular or oval shape has helped reduce the likelihood of ischemia, but this risk still remains under any circumstance in which the brain tissue is compressed.

While various brain access ports are known, there is an ongoing need to improve the art of surgical access ports.

### SUMMARY

In one exemplary aspect, there is provided an expandable surgical access port having an arm mounting body, a number of primary arms, a number of secondary arms, an activation ring, and an expandable membrane. The arm mounting body defines an arm mounting body opening surrounding a longitudinal axis, and the arm mounting body has a number of primary pivot locations surrounding the longitudinal axis and fixed at a respective first pivot location distance from the longitudinal axis, and a number of secondary pivot locations surrounding the longitudinal axis and fixed at a respective second pivot location distance from the longitudinal axis. The primary arms are arranged around the longitudinal axis, and each primary arm is pivotally attached at a respective primary pivot location and extends in a distal direction along the longitudinal axis to a respective primary arm distal end. The secondary arms are arranged around the longitudinal axis, and each secondary arm is pivotally attached at a respective secondary pivot location and extends in the distal direction along the longitudinal axis to a respective secondary arm distal end. The activation ring is rotatably mounted to the arm mounting body to rotate about the longitudinal axis between a first activation ring position and a second activation ring position. The activation ring has: a number of primary cam surfaces configured to each engage a respective primary arm to drive the respective primary arm from a respective closed position in which the respective primary arm distal end is a respective first arm distance from the longitudinal axis to a respective open position in which the respective primary arm distal end is a respective second arm distance from the longitudinal axis, upon movement of the activation ring from the first activation ring position to the second activation ring position, and a number of secondary cam surfaces configured to each engage a respective secondary arm to drive the respective secondary arm from a respective closed position in which the respective secondary arm distal end is a respective third arm distance from the longitudinal axis to a respective open position in which the respective secondary arm distal end is a respective fourth arm distance from the longitudinal axis, upon movement of the activation ring from the first activation ring position to the second activation ring position. The respective fourth arm distance of each secondary arm distal end is greater than the respective second arm distance of each primary arm distal end. The expandable membrane extends from a proximal membrane position, to a distal membrane position adjacent to each primary arm distal end and each secondary arm distal end. When the activation ring is in at least the second activation ring position, an entirety of the expandable membrane between the proximal membrane position and the distal membrane position comprises an elongated membrane cross-section as viewed along the longitudinal axis, the elongated membrane cross-section extending along a major membrane axis and a minor membrane axis perpendicular to the major membrane axis.

### BRIEF DESCRIPTION OF THE DRAWINGS

FIG. 1 is an isometric view of an exemplary embodiment of an expandable access port shown assembled with a navigation probe.

FIGS. 2A and 2B are side plan and isometric views of another exemplary embodiment of an expandable access port, shown with its expandable port in a contracted position.

FIGS. 3A and 3B are side plan and isometric views of the expandable access port of FIGS. 2A and 2B, shown with its expandable port 104 in an expanded position.

FIGS. 4A and 4B are top and bottom exploded isometric views of an activation assembly.

FIGS. 5A to 5D are bottom plan, top plan, side cross-section and front cross-section various views, respectively, of an exemplary port housing.

FIGS. 6A to 6D are bottom plan, top plan, side cross-section and front cross-section various views, respectively, of an exemplary lock ring.

FIGS. 7A to 7D are bottom plan, top plan, side cross-section and front cross-section various views, respectively, of an exemplary activation ring.

FIGS. 8A and 8B are top plan and side cross-section views, respectively, of an exemplary activation ring cover.

FIGS. 9A to 9D are top isometric, bottom isometric, side cross-section and front cross-section views, respectively, of an exemplary.

FIGS. 10A to 10D are top isometric, bottom isometric, detail side cross-section and top plan views, respectively, of an exemplary.

FIGS. 11A to 11C are top isometric, bottom isometric, and side cross-section views of an exemplary introducer.

FIG. 12 is a detail side cross-section view of the distal end of an assembled expandable access port.

FIGS. 13 and 14 are side cross-section views of an assembled expandable access port shown with the expandable port in the contracted position and expanded position, respectively.

FIGS. 15A to 15C are bottom isometric, top isometric, and side views, respectively, of another example of a port housing.

FIGS. 16A-16C are bottom isometric, top isometric and bottom plan views, respectively, of an exemplary connector housing.

FIGS. 17A and 17B are top isometric and bottom isometric views, respectively, of another example of an activation ring.

FIGS. 18A and 18B are detail side cross-section views of the proximal end of an assembled expandable access port, shown along two different cross-section planes.

FIGS. 19A to 19C are top plan, top isometric and bottom isometric views, respectively, of an exemplary surgical tool mount.

FIGS. 20A and 20B are front and side views of an exemplary guidance arm.

FIGS. 21A and 21B illustrate another example of a guidance arm and its connection to an expandable access port.

FIGS. 22A and 22B illustrate another example of a navigation probe lock.

FIG. 22C is a cross-section view of the navigation probe lock of FIGS. 22A and 22B.

FIGS. 23A and 23B illustrate the navigation probe lock of FIGS. 22A and 22C installed with an expandable access pot.

FIGS. 24A and 24B illustrate another example of a surgical tool mount.

FIG. 25 shows the surgical tool mount of FIGS. 24A and 24B attached to other parts of an expandable access port.

FIG. 26 is a cross-section view of an exemplary introducer.

FIGS. 27A and 27B are top views of an embodiment of an expandable surgical access port shown in the closed position and opened position, respectively.

FIG. 28A is a composite cross-section view as viewed along line 28A-28A showing the configuration of FIG. 27A on the left, and the configuration of FIG. 27B on the right.

FIG. 28B is a composite cross-section view as viewed along line 28B-28B showing the configuration of FIG. 27A on the left, and the configuration of FIG. 27B on the right.

FIGS. 29A and 29B are bottom views of the embodiment of FIGS. 27A to 28B, shown in the closed position and opened position, respectively.

FIGS. 30A and 30B are top views of an embodiment of an expandable surgical access port shown in the closed position and opened position, respectively.

FIG. 31A is a composite cross-section view as viewed along line 31A-31A showing the configuration of FIG. 30A on the left, and the configuration of FIG. 30B on the right.

FIG. 31B is a composite cross-section view as viewed along line 31B-31B showing the configuration of FIG. 30A on the left, and the configuration of FIG. 30B on the right.

FIG. 32A is a composite cross-section view of another embodiment of an expandable surgical access port, showing primary arms in the closed position on the left and in the opened position on the right.

FIG. 32B is a composite cross-section view of the embodiment of FIG. 31A, showing secondary arms in the closed position on the left and in the opened position on the right.

FIGS. 33A and 33B are bottom views of the embodiment of FIGS. 32A and 32B, shown in the closed position and opened position, respectively.

FIGS. 34A and 34B are top views of another embodiment of an expandable surgical access port shown in the closed position and opened position, respectively.

FIG. 35A is a composite cross-section view as viewed along line 35A-35A showing the configuration of FIG. 35A on the left, and the configuration of FIG. 35B on the right.

FIG. 36B is a composite cross-section view as viewed along line 36B-36B showing the configuration of FIG. 35A on the left, and the configuration of FIG. 35B on the right.

FIG. 36 is a bottom view of another embodiment of an expandable surgical access port shown in the opened position.

FIGS. 37A and 37B are bottom views of another embodiment of an expandable surgical access port shown in the opened position and the closed position, respectively.

FIG. 38 is a bottom view of another embodiment of an expandable surgical access port shown in the opened position.

FIGS. 39-41 are top views of alternative embodiments of expandable surgical access ports having introducers.

FIGS. 42A, 42C and 42D are a top views of an embodiment of an activation ring of an expandable surgical access port.

FIG. 42B is a side view of a secondary cam surface insert.

FIGS. 43A-43C are top views of another embodiment of an expandable surgical access port, shown in three different operating positions.

In the drawings, features that are repeated in substantially identical form are in many cases designated at a single location within the drawings to preserve the clarity of the drawings. Like features are designated in different embodiments with like reference numbers.

### DESCRIPTION OF EXEMPLARY EMBODIMENTS

The present disclosure provides non-limiting examples of embodiments of expandable brain access ports. Specific details of these embodiments are provided to aid in understanding, but such details are not intended to limit the scope of any of the appended claims, except as specifically recited in the claims. It will also be understood that certain details not currently recited in the claims may be added to the claims in the future, particularly as it becomes apparent through consideration of prior art and other factors that these details provide a benefit over the known art.

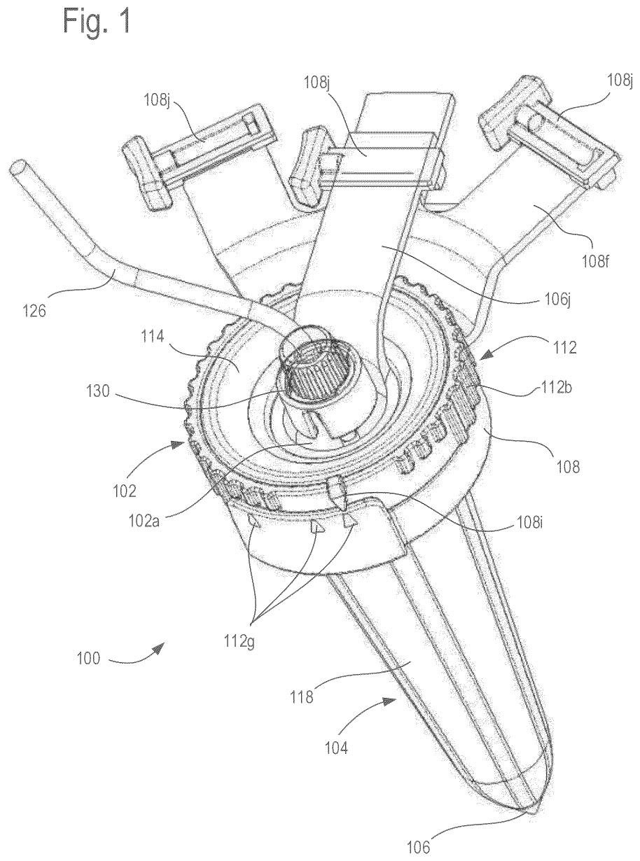

A first example of an expandable access port 100 is shown in FIG. 1. The expandable access port 100 generally includes an activation assembly 102, and expandable port 104, and an introducer 106. Details of these features are discussed below. The expandable access port 100 is configured to hold or provide access for one or more surgical instruments, such as endoscopes, resection tools, suction hoses, lights, and navigation devices. To this end, the activation assembly 102 defines an activation assembly opening 102a that surrounds a longitudinal axis 100a of the expandable access port 100.

In the case of FIG. 1, the expandable access port 100 is set up in a configuration to hold a navigation probe 126 located internal to the expandable access port 100. The navigation probe 126 is connected to a tracking system that monitors the position of the navigation probe 126. A typical navigation probe 126 and tracking system are registered to track the position of the probe's tip 126b and the trajectory of the probe shaft 126a. When integrated into the expandable access port 100, the probe tip 126b may be located at the distal tip of the introducer 106 to simply use the pre-set registration of the navigation probe 126 to track the introducer tip, and thus assume or extrapolate the position of the remainder of the expandable access port 100. It is also possible to register the navigation probe 126 to track the full shape and position of the expandable access port 100, regardless of the actual position of the probe tip 126b and probe shaft 126a. For example, if the probe tip 126b is seated a certain fixed distance from the distal end of the introducer 106, an offset can be programmed into the tracking system to account for this known offset. It is also possible to register the exact shape of the expandable access port 100 and/or the expandable port 104, to track the entire shape of device. Such navigation probes 126 and their tracking systems, including methods for setting an offset and determining the shape of the device, are known in the art, and need not be described in detail herein.

A probe lock 130 is provided to selectively hold the navigation probe 126 at a fixed location relative to the expandable access port 100. A non-limiting example of a suitable probe lock 130 may be found in U.S. application Ser. No. 17/473,282 (publication no. 2021/0401457), which is incorporated by reference herein. Other examples and details of probe locks 130 are provided below.

In use, the activation assembly 102 and expandable port 104 may be provided as an assembled structure that is not generally intended for disassembly (e.g., no reversible fasteners such as screws), but this is not strictly required. The introducer 106 can be secured to the activation assembly 102 and expandable port 104 to facilitate atraumatic insertion of the expandable access port 100 into the brain to the surgery site. The navigation probe 126 may be used to help direct the expandable access port 100 precisely to the surgery site. Upon insertion to the desired location, the introducer 106 is removed, and the activation assembly 102 is operated to expand the expandable port 104. At the conclusion of surgery, the expandable access port 100 is withdrawn from the brain. The expandable port 104 may or may not be retracted prior to withdrawing the expandable access port 100.

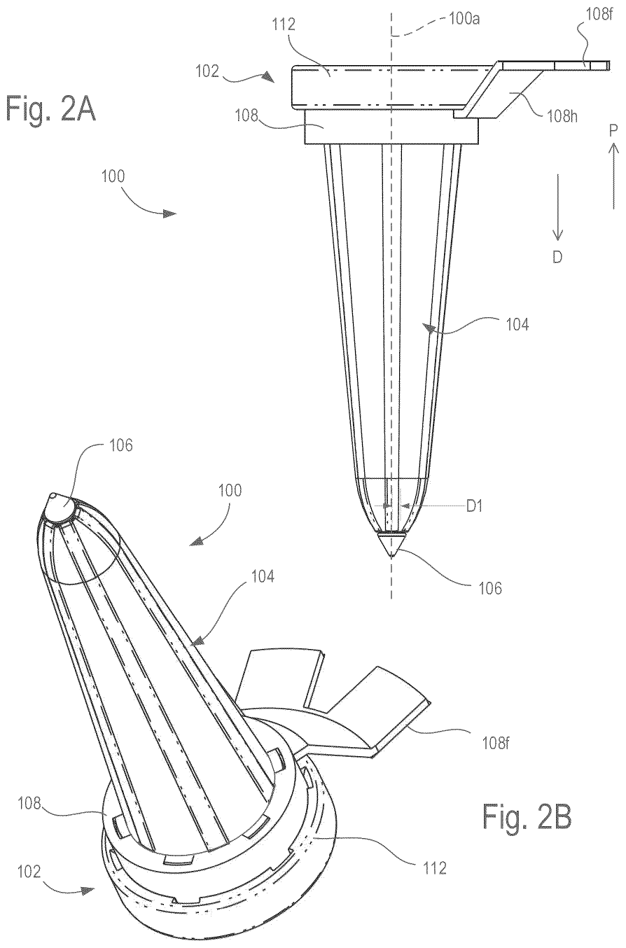

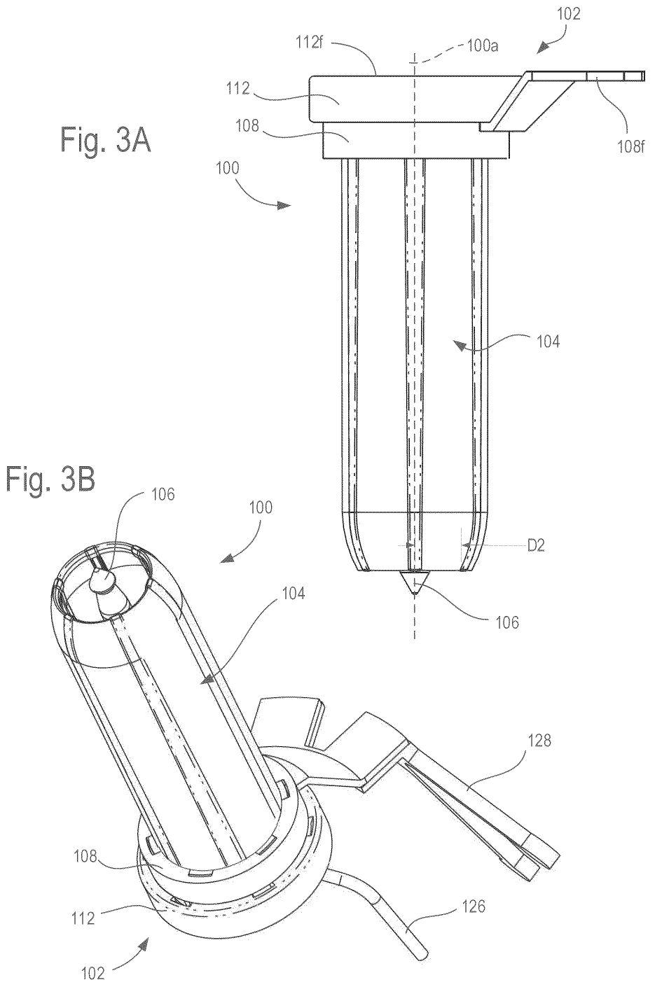

FIGS. 2A-3B show another embodiment of an expandable access port 100, which is similar to the embodiment of FIG. 1, but with certain alternative structures as described below. FIGS. 2A and 2B show the expandable access port 100 with the expandable port 104 in the contracted position, and FIGS. 3A and 3B show the expandable port 104 in the expanded position. In both cases, the introducer 106 is assembled to the remainder of the expandable access port 100. As shown in FIG. 2A, the expandable port 104 extends along the longitudinal axis 100a in a distal direction D from the activation assembly 102. To help in explaining features herein, FIG. 2A also illustrates a proximal direction P, which is opposite the distal direction D.

FIG. 3B also shows a navigation probe 126 in place inside the introducer 106, and a guidance arm 128 mounted to the activation assembly 102. The guidance arm 128 also serves as a tracking device to directly monitor the position of the expandable access port 100 after the navigation probe 126 is removed, or if the device is used without a navigation probe 126. Details of the guidance arm 128 are provided below.

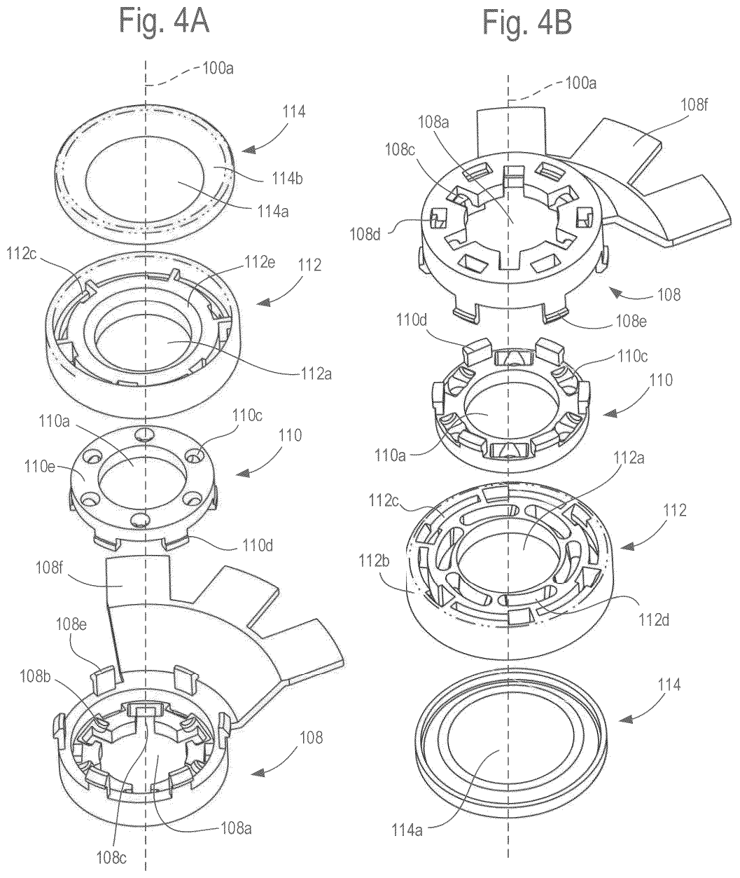

An exemplary embodiment of an activation assembly 102 is shown in FIGS. 4A-8B. The activation assembly 102 generally includes a port housing 108 and a lock ring 110 that are assembled together to form an activation arm mounting body, and an activation ring 112 that is movably mounted to the activation arm mounting body. The port housing 108 defines a port housing opening 108a, the lock ring 110 defines a lock ring opening 100a, and the activation ring 112 defines an activation ring opening 112a. The port housing opening 108a, lock ring opening 100a and activation ring opening 112a are aligned, and each surrounds or defines at least a portion of the activation assembly opening 102a.

In this example, the activation ring 112 is rotationally mounted to the activation arm mounting body, such as explained below, such that the activation ring 112 can be rotated relative to the activation arm mounting body and about the longitudinal axis 100a, between a first activation ring position and a second activation ring position. The first activation ring position is shown in FIGS. 2A and 2B, and in this position the expandable port 104 is in the contracted position. The second activation ring position is shown in FIGS. 3A and 3b, and in this position the expandable port 104 is in the expanded position.

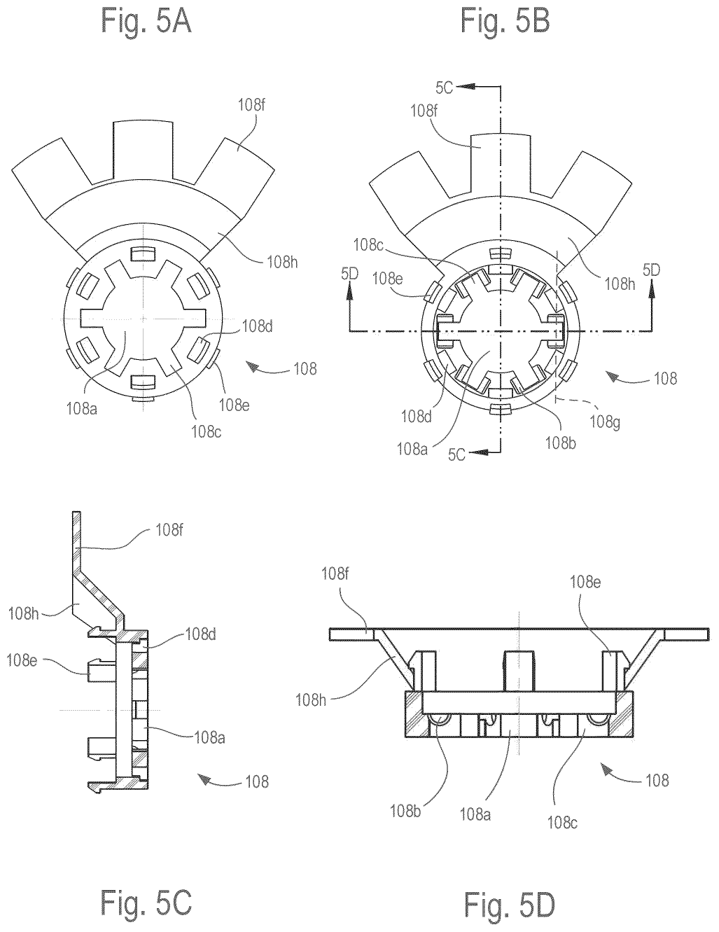

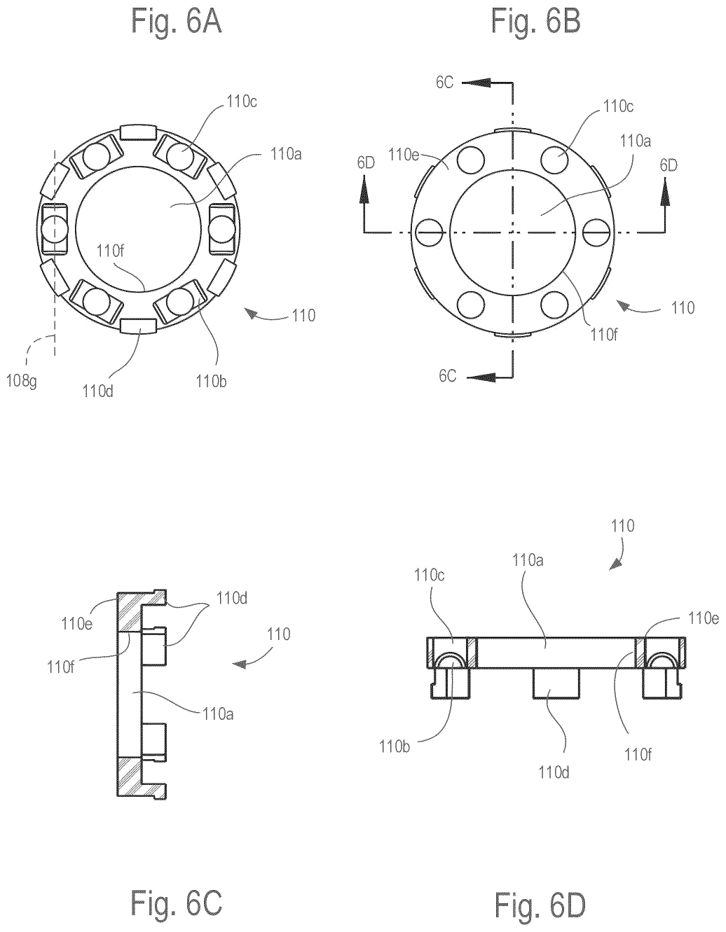

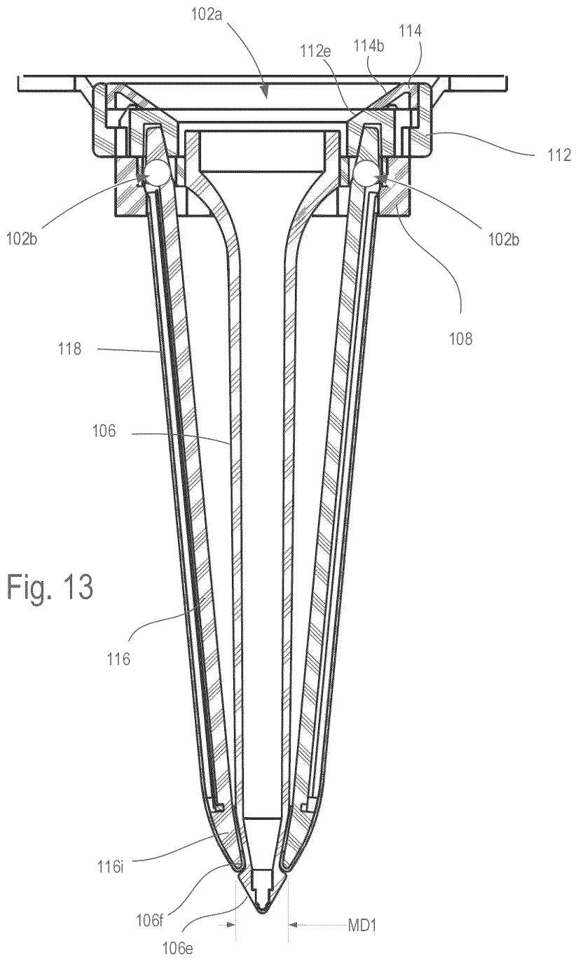

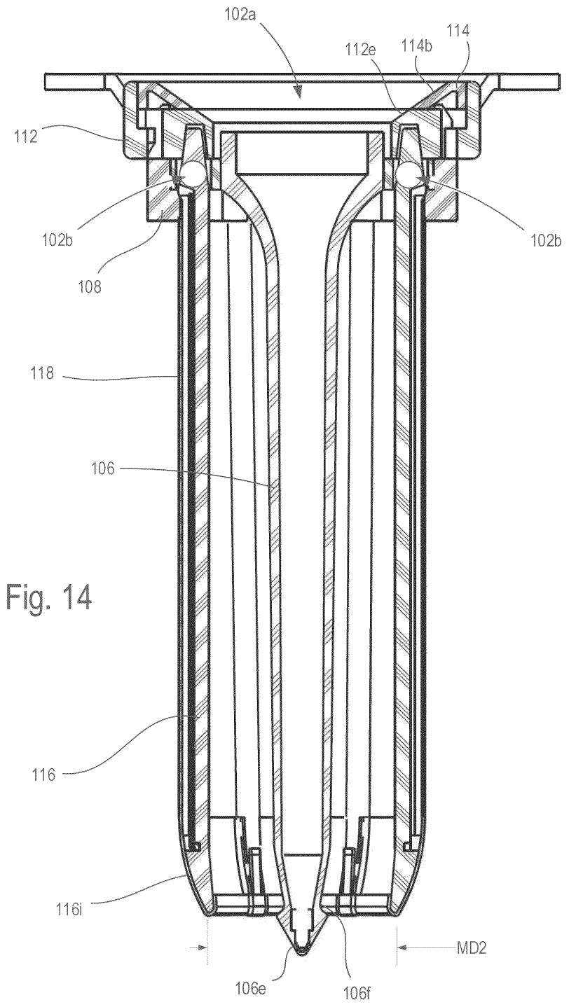

As best shown in FIGS. 4A-5D, the port housing 108 comprises a generally ring-shaped structure that defines the port housing opening 108a. The port housing 108 includes a plurality of first pivot recesses 108b that surround the longitudinal axis 100a. Similarly, the lock ring 110 is a generally ring-shaped structure that defines the lock ring opening 100a, and includes a plurality of second pivot recesses 110b that surround the longitudinal axis 100a. The port housing 108 and lock ring 110 are secured together, such that the first pivot recesses 108b and second pivot recesses 110b collectively form cavities that each define a respective pivot locations 102b (see FIGS. 13 and 14). The pivot locations 102b may be cylindrical (as shown), spherical, or have any other shape suitable to hold a corresponding pivot to rotate about a fixed axis. The pivot locations 102b are distributed around the longitudinal axis 100a, and each defines a respective pivot axis 108g (see FIGS. 5B and 6A). The pivot axes 108g lie in a common plane that is perpendicular to the longitudinal axis 100a. The pivot axes 108g are oriented such that they do not intersect the longitudinal axis 100a. Each pivot axis 108g also preferably extends tangentially to the longitudinal axis 100a (i.e., tangential to an imaginary circle having its center at the longitudinal axis 100a, with each pivot axis 108g being equidistant at its closest location to the longitudinal axis 100a. The pivot axes 108g also may be equidistantly positioned in the circumferential direction, such as shown in FIGS. 5B and 6A, which show six pivot axes 108g spaced at angles of 60° about the longitudinal axis 100a.

The lock ring 110 is secured to the port housing 108 by any suitable means. For example, the lock ring 110 is secured to the port housing 108 by locking tabs 110d that snap into respective locking tab receivers 108d, preferably in a manner that does not readily facilitate disconnection. In the illustrated example, the locking tabs 110d are provided on the lock ring 110, and the locking tab receivers 108d are provided on the port housing 108, but this arrangement may be reversed in whole or in part (i.e., one more of the locking tab receivers 108d may be on the lock ring 110, and one or more of the locking tabs 110d may be on the port housing 108). In other cases, the lock ring 110 and port housing 108 may be connected by adhesives, ultrasonic welding, rivets, reversible mechanical fasteners (e.g., screws), and so on.

The port housing 108 and/or lock ring 110 may include supplemental structures to increase their utility. For example, the port housing 108 may include one or more extensions 108f to which the introducer 106 and accessories (surgical tools, navigation devices, etc.) may be mounted. The extensions 108f also may be configured to secure to a clamp to hold the expandable access port 100 at a fixed location relative to the patient, operating table or surgical frame. Each extension 108f may include a lock mechanism 108j, or be shaped to connect to a lock provided on a different part. The details of such locks are not the subject of this disclosure, are well known in the art, and need not be described herein.

The activation arm mounting body (the connected port housing 108 and lock ring 110) may include multiple extensions 108f (e.g., two to four extensions 108f). The extensions 108f also may be arranged on one side of the activation arm mounting body (i.e., all within a 180° segment, or more preferably a 90° segment, about the longitudinal axis 100a). This provides greater access for the surgeon to operate without obstruction on the other side of the activation arm mounting body.

As noted above, the activation ring 112 is secured to the activation arm mounting body to rotate about the longitudinal axis 100a relative to the activation arm mounting body. In this example, the port housing 108 includes a plurality of sliding tabs 108e that snap into corresponding sliding tab receivers 112c in the activation ring 112. Each sliding tab receiver 112c comprises a circumferential slot having a relatively narrow width in the radial direction, and an end portion having a somewhat larger width in the radial direction. Each sliding tab 108e terminates at a hook that can be inserted into the wide end portion of each sliding tab receiver 112c, and then slid along the narrow portion of the sliding tab receiver 112c to rotate the activation ring 112 relative to the port housing 108. A hook or protrusion (not shown) may be provided between the wide and narrow portions of each sliding tab receiver 112c to prevent the respective sliding tab 108e from returning to the wide portion of the sliding tab receiver 112c. Thus, the parts cannot be disassembled accidentally during use, and more preferably cannot be disassembled under any normal circumstances (e.g., without breaking the parts).

In the illustrated example, the sliding tabs 108e are provided on the port housing 108, and the sliding tab receivers 112c are provided on the activation ring 112, but this arrangement may be reversed in whole or in part (i.e., one more of the sliding tabs 108e may be on the activation ring 112, and one or more of the sliding tab receivers 112c may be on the port housing 108). Also, the sliding tabs 108e and/or sliding tab receivers 112c may be provided on the lock ring 110, rather than the port housing 108.

In other embodiments, the activation ring 112 may be rotationally fixed to the activation arm mounting body using other connections. For example, the activation ring 112 may be captured in place against the activation arm mounting body by a central locking ring that threads into the activation arm mounting body. As another example, pins or screws may be inserted through the sliding tab receivers 112c and secured to the activation arm mounting body to capture the activation ring 112 in place. Other alternatives and embodiments will be apparent to persons of ordinary skill in the art in view of the present disclosure.

In still other embodiments, the activation ring 112 may be mounted to the activation arm mounting body move in other directions rather than the rotation described above. For example, the activation ring 112 may be mounted on rails to slide laterally relative to the activation arm mounting body.

Referring to FIG. 2A (as an example), the activation ring 112 preferably is located on the proximal side of the port housing 108. This allows unhindered access to the activation ring 112 when the activation arm mounting body is secured in place at the surgery site. Furthermore, the arrangement of sliding tabs 108e and sliding tab receivers 112c allows the activation ring 112 to rotate relative to the activation arm mounting body throughout its entire operative range of motion while remaining at a fixed location along the longitudinal axis longitudinal axis 100a. This arrangement minimizes the overall length of the expandable access port 100, and prevents the possible creation of unwanted axial forces along the longitudinal axis 100a as the activation ring 112 is rotated relative to the activation arm mounting body. While preferred, this arrangement is not required in all embodiments.

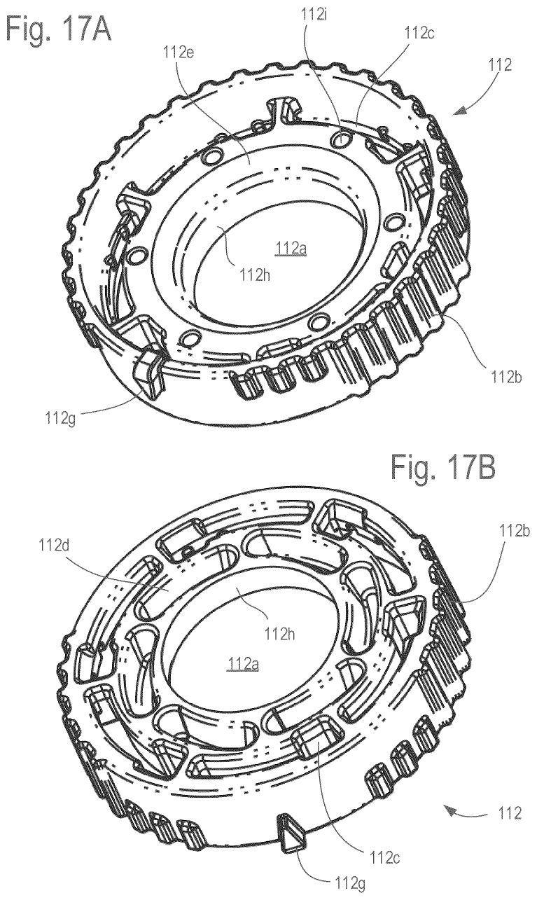

As also shown in FIGS. 1, 17A and 17B, the activation ring 112 may have an outer surface defining a grip 112b, and the grip 112b optionally may be larger in diameter than an adjacent portion of the activation arm mounting body. This also facilitates ease of use, by providing a tactile distinction between the activation ring 112 and activation arm mounting body, and helping to ensure that rotational forces are not erroneously applied to the activation arm mounting body during operation, and increasing the surgeon's ability to firmly hold the activation ring 112.

As shown in FIG. 2A, one or more of the extensions 108f may extend to be positioned at, or spaced in the proximal direction from, a proximal side 112f of the activation ring 112. For example, one or more of the extensions 108f may be secured to the ring-like portion of the port housing 108 via an extension base 108h that extends in the proximal direction P. This arrangement makes the extensions 108f more accessible for connecting to accessories and position locks, and allows the expandable port 104 to be positioned deeper in the brain.

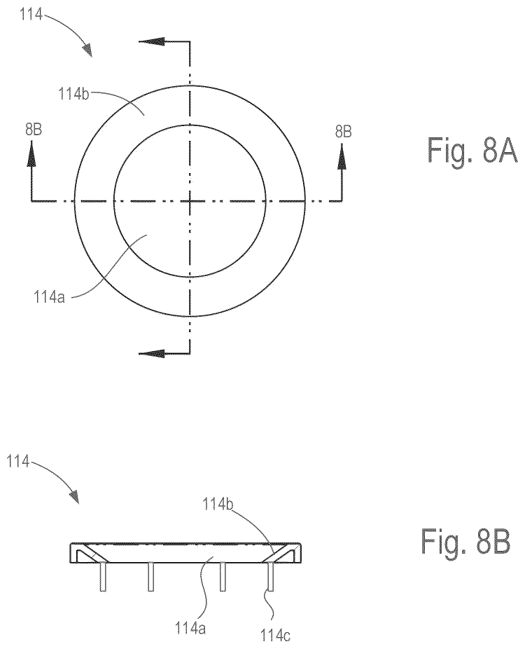

Referring to FIGS. 8A-8B, the activation assembly 102 may include an activation ring cover 114 that covers the proximal side 112f of the activation ring 112. The activation ring cover 114 encloses the proximal sides of the sliding tab receiver 112c to prevent ingress of liquids or other matter that might obstruct operation of the activation ring 112. The activation ring cover 114 has an activation ring cover opening 114a that is concentric with the activation ring opening 112a. As best shown in FIGS. 13 and 14, the activation ring 112 may define a first tapered inlet surface 112e, and the activation ring cover 114 may define a second tapered inlet surface 114b that align to form a continuous tapered entry to the activation assembly opening 102a that decreases in diameter in the distal direction D. The activation ring cover 114 may be secured to activation ring 112 by any means, such as adhesives, snap fitment, ultrasonic welding, and so on. In this example, the activation ring cover 114 has pins 114c that are secured into holes 112i (see Figure in the adjacent face of the activation ring 112. One or all of the activation ring cover 114, first tapered inlet surface 112e and activation ring cover opening 114a also may be omitted.

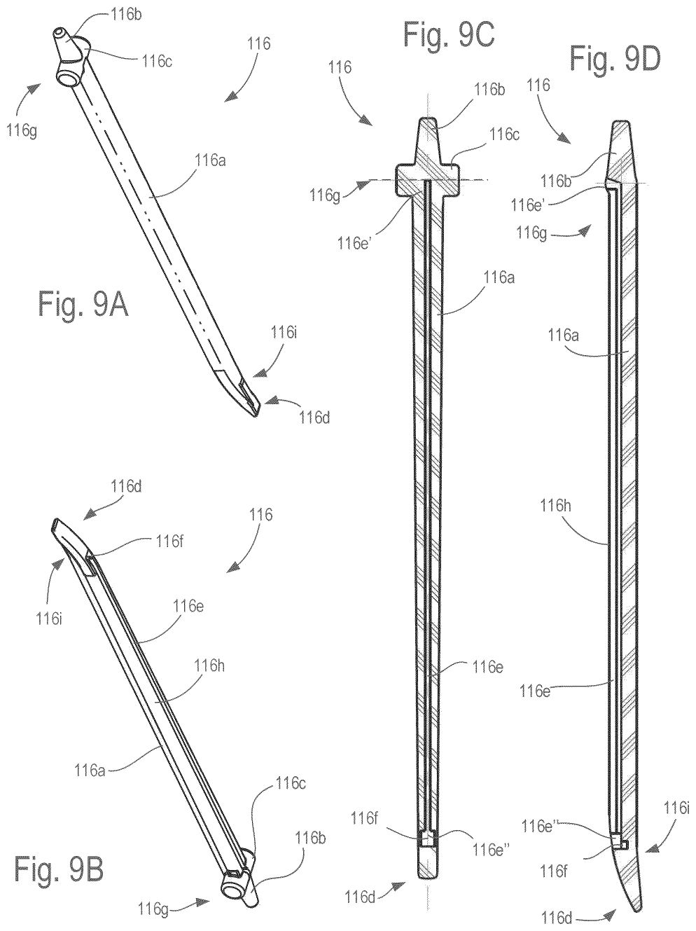

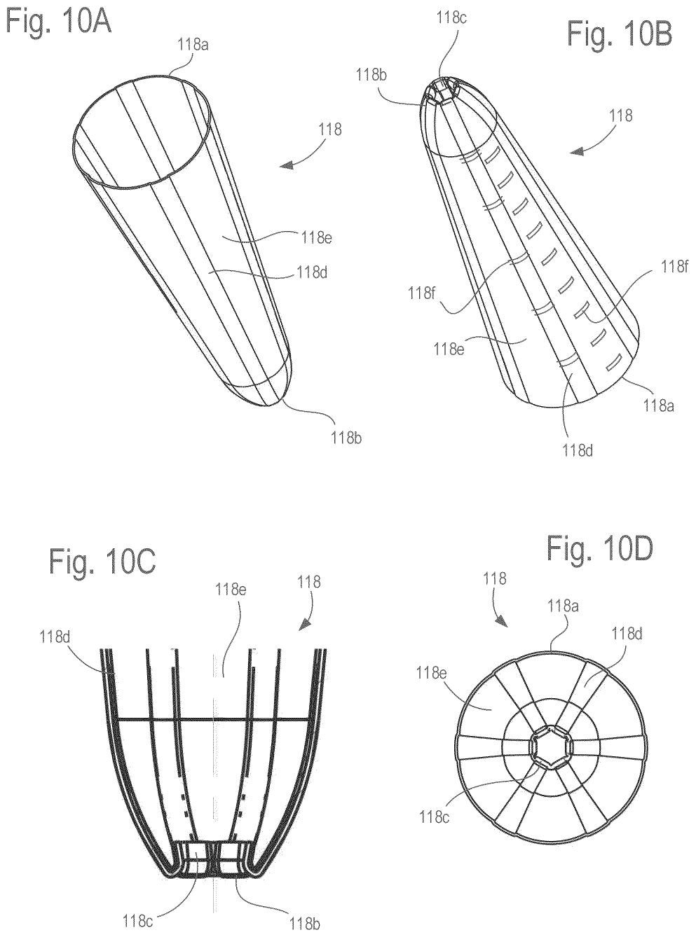

Details of the expandable port 104 are now described in relation to FIGS. 9A-10D. The expandable port 104 generally includes a plurality of activation arms 116 and a membrane 118.

Each activation arm 116 comprises an elongated body 116a that extends in the distal direction D from a respective proximal arm end 116g to a respective distal arm end 116d. The proximal arm end 116g of each activation arm 116 is pivotally attached to the activation arm mounting body by a respective pivot 116c. In this example, each pivot 116c comprises a cylindrical body that is captured in place at a respective pivot location 102b and extends along the respective pivot axis 108g when the activation arm 116 is assembled to the activation arm mounting body (i.e., when the pivot 116c is captured between a respective first pivot recesses 108b and a respective second pivot recess 110b). This allows the activation arm 116 pivot about a respective pivot axis 108g. In other cases, the pivots 116c may comprise spherical bodies, pins that are provided separately and inserted through holes in the activation arm 116, and so on.

Each activation arm 116 is pivotable between a first arm position, in which the distal arm end 116d is a first distance D1 from the longitudinal axis 100a (see FIG. 2A), and a second arm position, in which the distal arm end 116d is a second distance D2 from the longitudinal axis 100a (see FIG. 3A). The second distance D2 is greater in magnitude than the first distance D1. The activation ring 112 is operable to move the activation arms 116 between their respective first and second arm position. More specifically, the activation ring 112 is rotatable between a first activation ring position in which the activation arms 116 are in their first arm positions, and a second activation ring position in which the activation arms 116 are in their second arm positions.

The activation arms 116 preferably cannot be moved any closer together than the first distance D1, so as to prevent the activation arms 116 from pinching brain tissue when the activation arms 116 are contracted without the presence of the introducer 106. This may be achieved, for example, by configuring the activation ring 112 such that it cannot move the activation arms 116 inwardly beyond the first position, by providing travel stops that contact the activation arms 116, or by making the activation arms such that they converge to contact each other along their circumferential sides in the first position. Other alternatives and embodiments will be apparent to persons of ordinary skill in the art in view of the present disclosure.

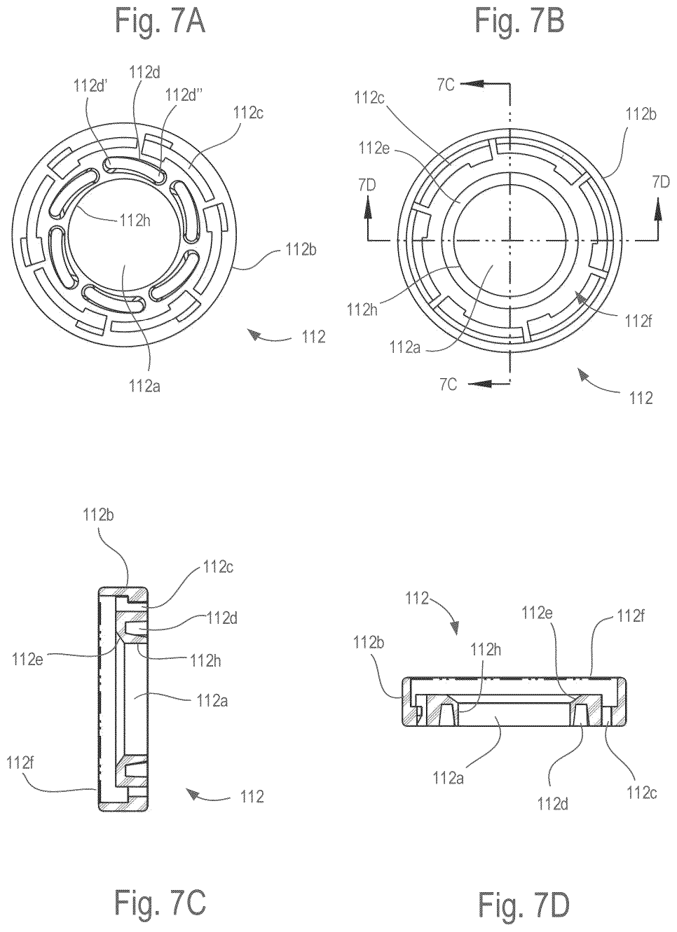

In this example, the activation ring 112 moves the activation arms 116 by engagement between respective cam slots 112d in the activation ring 112, and respective cam followers 116b at the proximal arm ends 116g of each slot 116e. As shown in FIG. 7A, the cam slots 112d are formed as recesses in the lower face of the activation ring 112. As the name suggests, each cam slot 112d is formed as an eccentric ramp about the longitudinal axis 100a. Specifically, each cam slot 112d extends about a portion of the activation ring opening 112a from a respective first cam slot end 112d′ to a respective second cam slot end 112d″, and each respective second cam slot end 112d″ is closer to the longitudinal axis 100a than each respective first cam slot end 112d′. The portion of the cam slot 112d between the first cam slot end 112d′ and the second cam slot end 112d″ may be straight, arced (shown) or have any other smooth continuous shape that performs the function described herein.

Each cam follower 116b extends into a respective one of the cam slots 112d, and rotating the activation ring 112 relative to the activation arm mounting body causes the cam slots 112d to drive the respective cam follower 116b towards or away from the longitudinal axis 100a, depending on the direction of rotation. In this case, each activation arm 116 acts as a class 1 lever, with the pivot 116c located between the cam follower 116b and the distal arm end 116d. Thus, when the cam followers 116b are located at the first cam slot ends 112d′ the distal arm ends 116d are located in their respective first arm positions to contract the expandable port 104, and when the cam followers 116b are located at the second cam slot ends 112d′ the distal arm ends 116d are located at their respective second arm positions to expand the expandable port 104. The cam slots 112d may include protrusions (not shown) that extend inwardly to provide one or more locations at which movement of the respective cam followers 116b is inhibited without applying a somewhat greater torque to the activation ring 112. Such protrusions can be positioned to establish predefined locations at which the activation arms 116 are held at one or more positions. For example, a protrusion may be provided to hold the activation arms 116 at their respective first or second positions, or anywhere between. Such protrusions can help the surgeon feel where the determined locations are. Other shapes, such as bends in the cam slots 112d can be provided to serve the same function.

The cam slots 112d may be shaped such that the cam followers 116b cannot back-drive the activation ring 112. Specifically, the angle of contact between the cam followers 116b and cam slots 112d may be selected such that a force applied to rotate the arm 116 generates a frictional load that prevents relative motion between the parts. This angle can be determined using conventional engineering principles (e.g., static coefficient of friction of an object on a ramp), and need not be describe in further detail herein.

The cam slots 112d also may be shaped to capture both sides the cam followers 116b such that the arms 116 cannot move freely in either direction. This prevents the arms 116 from moving beyond the position dictated by one side of the cam slot surface 112, and provides precise control of the arms' positions when rotating the activation ring 112 in alternating directions. This is expected to be beneficial to allow the surgeon to apply driving forces to precisely open and close the arms 116, preferably to any desired position, without relying on resilient forces (e.g., pressure from brain tissue) to collapse the arms 116 when it is desired to retract the arms. The cam slots 112d preferably also are configured to prevent the activation ring 112 from being moved to drive the activation arms 116 beyond their respective first position, such as by terminating each cam slot 112d at a closed end that stops on the cam follower 116b.

In other embodiments, the activation ring 112 can be configured to operate as a class 2 lever on the activation arms 116. For example, the cam slots 112d and cam followers 116b may be located between the pivot 116c and distal arm end 116d of each 116. Other alternatives and embodiments will be apparent to persons of ordinary skill in the art in view of the present disclosure.

The activation arm mounting body is configured to permit engagement between the cam slots 112d and cam followers 116b, such as by including a respective cam follower port 110c passing through the lock ring 110 to a proximal side 110e of the lock ring 110 to accommodate each cam follower 116b. Similarly, the activation arm mounting body is configured to permit the activation arms 116 to rotate distally from each pivot location 102b, such as by providing a respective activation arm port 108c on the distal side of each pivot location 102b. The cam follower ports 110c and/or activation arm ports 108c may be dimensioned to prevent excessive motion of the activation arms 116 if the activation ring 112 becomes detached from the activation arm mounting body.

In the shown example, the mechanism is configured such that all of the activation arms 116 move in unison at all times, thus ensuring that the device maintains a uniform generally circular shape during opening and closing. However, other embodiments may have features for adjusting the movement of, or disabling, one or more activation arms 116.

The membrane 118 surrounds the activation arms 116, and extends from a proximal membrane end 118a adjacent the activation assembly 102 to a distal membrane end 118b adjacent to the distal arm ends 116d. The membrane 118 comprises a flexible material that is expandable to permit the distal arm ends 116d to move from their respective first positions to their respective second positions. The membrane 118 may be secured to the activation arms 116, but preferably is overmolded onto the activation arms 116. Overmolding can be accomplished by placing the assembled activation assembly 102 and activation arms 116 into a mold that receives the activation arms 116, and injecting the membrane material into the mold to surround each activation arm.

The membrane 118 may comprise any suitable material that provides the desired degree of elongation. For example, the membrane 118 may comprise a thermoplastic elastomer, and/or an elastomer based on styrenic olefinic rubber and hydrogenated isoprene, containing polypropylene as a reinforcing agent and mineral oil as a plasticizer and processing aid.

As shown in FIG. 10D, the membrane 118 may be molded to have a generally circular cross-sectional profile, as viewed along the longitudinal axis 100a. The membrane 118 also may be shaped to have a respective distinct rib 118d located at each activation arm 116, and a respective wall 118e located between each adjacent pair of activation arms 116. As shown in FIG. 10D, each rib 118d may have a slightly larger diameter than the adjacent walls 118e, but this is not required. The ribs 118d may be molded to fully surround each 116, or they may only surround the respective outer radial surface 116h (i.e., the side facing away from the longitudinal axis 100a) of each 116. The membrane 118 may extend fully from the activation assembly 102 to the distal arm ends 116d, and may include a lip 118c that wraps around the distal arm ends 116d.

The membrane 118 also may include depth markers 118f (see FIG. 10B), which may be printed onto the membrane 118 or formed as bumps or protrusions. The depth markers 118f may include numerical characters (e.g. numbers indicating dimensions or relative locations) or other shapes (e.g., letters) to identify a respective position of each depth marker 118f. The depth markers also may comprises printed images or bumps/protrusions defined on the activation arms 116, that are visible through the membrane 108. Other alternatives and embodiments will be apparent to persons of ordinary skill in the art in view of the present disclosure.

The membrane 118 may have any suitable dimensions. For example, the membrane 118 may have a wall thickness of 0.024 inches to 0.008 inches, and more preferably of inches to 0.012 inches, and even more preferably of 0.015 inches to 0.017 inches. In one embodiment, the membrane 118 may have a nominal wall thickness of 0.016 inches. It will be appreciated that these, and other dimensions herein, are subject to manufacturing tolerances, and the recitation of a specific number is intended to include typical variations due to manufacturing tolerances.

The membrane 118 also may be selected to provide a desirable degree of expansion to allow the activation arms 116 to open to the desired second arm positions. For example, the membrane 118 may be selected such that it expands by at least 250% of its original circumference at the point of greatest elongation (typically the distal membrane end 118b). More preferably, the membrane 118 may be selected such that it expands by at least 300%, and even more preferably by 350% at the point of greatest elongation. This expansion is illustrated in FIGS. 13 and 14 as the change between membrane diameter MD1 at the distal membrane end 118b in FIG. 13 and the membrane diameter MD2 at the distal membrane end 118b in FIG. 14.

The membrane 118 also may have and suitable size for use as a brain retractor. For example, the membrane 118 may have a contracted diameter of MD1 of 0.40 inches and an expanded diameter of 0.80 inches or more. In other cases, the membrane 118 may have a contracted diameter of MD1 of 0.30 inches and an expanded diameter of 0.90inches or more. In still other cases, the membrane 118 may have a contracted diameter of MD1 of 0.25 inches and an expanded diameter of 1.00 inches or more.

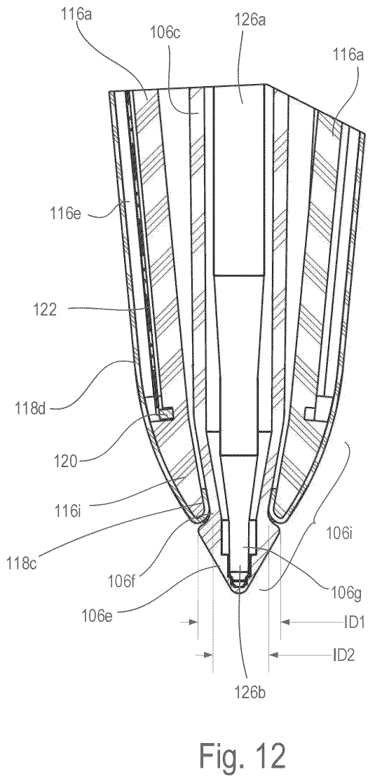

Referring back to FIGS. 9A-9D, and also to FIG. 12, one or more of the activation arms 116 may include a light 120. The light 120 may comprise a light emitting diode (LED), a terminal end of a light guide (e.g., a fiber optic cable), and so on. The activation arm 116 also may be formed as a light guide that is optically connected to a remote light source. In the shown example one or more of the activation arms 116 is formed of a transparent material (e.g., polycarbonate plastic), and has an LED light 120 located adjacent the distal arm end 116d. Light from the light 120 can pass through the distal arm end 116d to reach the surgery site. The distal arm end 116d also may be shaped or have surface treatments that help guide and distribute the light at the surgery site. Such shapes (e.g. Fresnel-type lenses or pyramidal bumps) and surface treatments for guiding and distributing light are known in the art.

The light 120 may be mounted to an inner surface of the activation arm 116 or at other locations. Preferably, the light 120 is mounted in a light receiver 116f that is recessed into the activation arm 116. The activation arm 116 has a slot 116e that leads to the light receiver 116f. The slot 116e is dimensioned to receive a light connector 122, such as a light guide or an electrical wire to power the light 120. In the shown example, the slot 116e extends along the activation arm 116 from a proximal slot end 116e′ to a distal slot end 116e″ adjacent the light receiver 116f. The proximal slot end 116e′ may be located at or near the pivot 116c, or at any other location where access may be provided for a light connector 122 to enter the slot 116e.

The slot 116e may be located at any part of the activation arm 116, but preferably extends along the outer radial surface 116h. In this case, the membrane 118 can be overmolded over the outer radial surface 116h, such that a portion of the rib 118d is overmolded into the slot 116e. In this case, the engagement between the membrane 118 and the outer radial surface 116h helps hold the rib 118d at a fixed location as the activation arm 116 moves to its second (expanded) position. When fully constructed, the membrane 118 also encases the light 120 and the light connector 122 between the outer radial surface 116h and 118, thus holding the light 120 in place during use and keeping the light 120 away from contact with the brain tissue.

It will also be appreciated that one or more of the activation arms 116 may include a slot 116e or other shapes for the purpose of receiving an overmolded part of the membrane 118 (i.e., without a light 120), to enhance the connection between the activation arms 116 and the membrane 118.

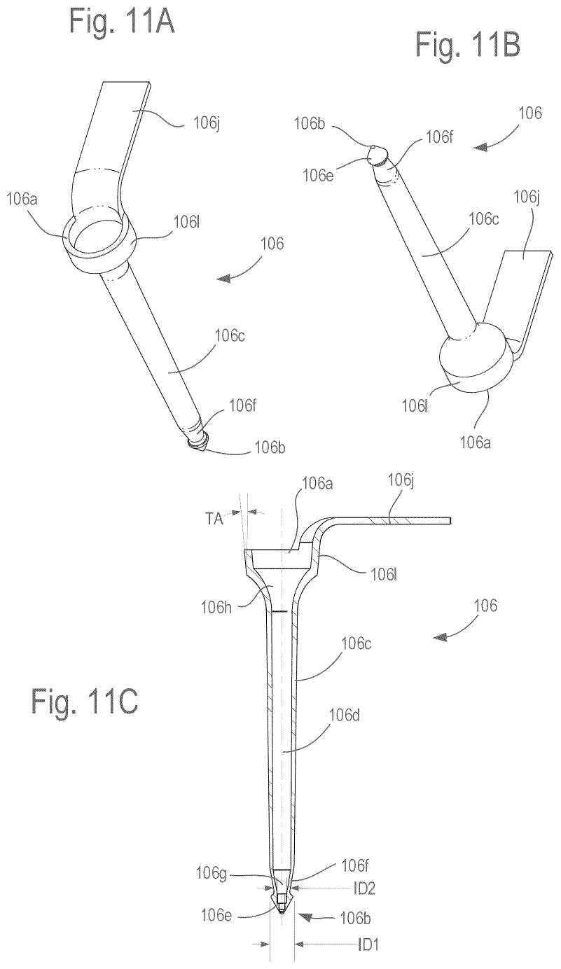

Referring now to FIGS. 11A-14, an exemplary introducer 106 and its interaction with the remainder of the expandable access port 100 is described in detail. The introducer 106 extends from a proximal introducer end 106a to a distal introducer end 106b, and has a tubular wall 106c that defines a cannula 106d. The cannula 106d extends along the longitudinal axis 100a from the proximal introducer end 106a to a point adjacent to the distal introducer end 106b. The cannula 106d terminates at a probe tip receiver 106g that is configured to receive one or more different navigation probes 126. When fully inserted, the probe shaft 126a extends along the cannula 106d, and the probe tip 126b seats in the probe tip receiver 106g to hold the probe tip 126b at a fixed location.

At the distal introducer end 106b, the introducer 106 has an introducer tip 106e, which tapers to increase in size in the proximal direction P, to a diameter ID1. The introducer tip 106e optionally may have an opening that leads into the probe tip receiver 106g, which can be helpful to vent pressure in the brain as the expandable access port 100 is inserted. On the proximal side of the introducer tip 106e, the introducer 106 has an outer annular recess 106f. The annular recess 106f is region of the tubular wall 106c that has a reduced diameter ID2 as compared to the maximum diameter ID1 of the introducer tip 106e.

The introducer 106 is connected to the remainder of the expandable access port 100 by inserting it through the activation assembly opening 102a, and securing the proximal introducer end 106a to the activation arm mounting body. In this case, the introducer 106 has a mounting tab 106j that extends radially from the tubular wall 106c to overlie and connect to one of the extensions 108f, to thus hold the introducer 106 in an operative position in which the expandable access port 100 can be inserted into the brain to the surgery site. In the operative position, the cannula 106d extends along the longitudinal axis 100a, and the introducer tip 106e extends in the distal direction D beyond the distal arm ends 116d. This arrangement is best shown in FIGS. 12 and 13.

With the introducer 106 in the operative position and the activation arms 116 in their respective first (contracted) positions, at least a portion of each distal arm end 116d is received within the annular recess 106f. This helps prevent the distal arm ends 116d from pulling on the brain tissue as the expandable access port 100 is inserted and prevents the brain tissue from pulling the activation arms 116 away from the introducer 106. Furthermore, each activation arm 116 also preferably includes an inward bend 116i at its distal arm end 116d, to help form a continuous tapered outer wall 106i extending from the distal introducer end 106b to a point along or behind the annular recess 106f. Each inward bend 116i comprises a portion of the respective activation arm 116 that is bent towards the longitudinal axis 100a to form a tapered portion of the outer radial surface 116h. In the shown example, the inward bend 116i is located at the end of a straight portion of the elongated body 116a. At least a portion of each inward bend 116i extends into the annular recess 106f, and the outer surface of the inward bend 116i (or portion of the membrane 118 surrounding the inward bend 116i) preferably forms a curved taper that transitions gradually between the taper angle of the introducer tip 106e and the taper angle of the straight portion of the outer radial surface 116h. However, it is also envisioned that the inward bend 116i may meet the slot 116e or the straight portion of the elongated body 116a at a distinct angle.

The foregoing arrangement provides several benefits. First, the continuous tapered outer wall 106i portion formed by the introducer tip 106e and inward bend 116i presents an atraumatic shape for inserting the expandable access port 100 into the brain. At the same time, the inward bends 116i can be made relatively wide, as compared to arms that extend straight to the introducer tip 106e, which helps increase the stiffness of the activation arms 116 at their distal arm ends 116d. Still further, the inward bends 116i also present a curving surface at the brain tissue when the expandable port 104 is expanded, such as shown in FIG. 14, which is expected to reduce the likelihood and/or severity of ischemia along the distal arm ends 116d and distal membrane end 118b.

The introducer 106 also may include other features to increase its utility. For example, the proximal introducer end 106a may be formed with a tapered inlet 106h to help guide a navigation probe 126 into the cannula 106d. The introducer 106 also may be formed to mate closely with the activation assembly 102 to hold the introducer 106 against movement perpendicular to the longitudinal axis 100a (i.e., lateral movement). For example, the introducer 106 may have an introducer outer face 106l that contacts a corresponding lock ring inner face 110f of activation ring inner face 112h to prevent lateral movement of the proximal introducer end 106a relative to the activation assembly 102. One of more of the introducer outer face 106l, lock ring inner face 110f and activation ring inner face 112h also may be tapered to decrease in size in the distal direction D. For example, the introducer outer face 106l and lock ring inner face 110f may have matching taper angles TA, or all three of the introducer outer face 106l, lock ring inner face 110f and activation ring inner face 112h may have matching taper angles. In this case, when the introducer 106 is assembled to the rest of the expandable access port 100, the matching taper angles prevent the introducer 106 from moving laterally relative to the rest of the expandable access port 100, and also hold the introducer 106 at a fixed location along the longitudinal axis 100a to prevent it from being inserted beyond the desired location. The matching taper angles also may inhibit or prevent relative rotation of the introducer 106, lock ring 110 and activation ring 112, to thereby prevent accidental rotation of the activation ring 112 as the expandable access port 100 is inserted into place.

It will be appreciated that a structure comparable to the lock ring inner face 110f may instead be provided at any other part of the activation assembly 102, such as by being formed as an inner surface wall or walls of the port housing 108 and/or activation ring cover 114. Other alternatives and embodiments will be apparent to persons of ordinary skill in the art in view of the present disclosure.

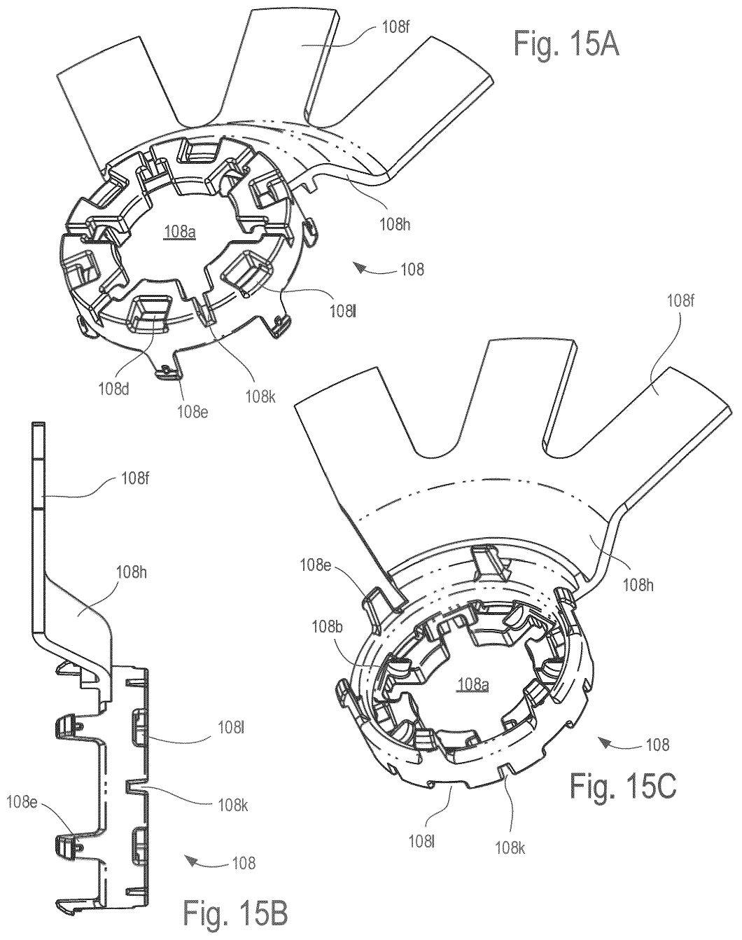

Other embodiments of certain features are shown in FIGS. 15A through 18B. These embodiments may be used together, or in combination with other embodiments described herein.

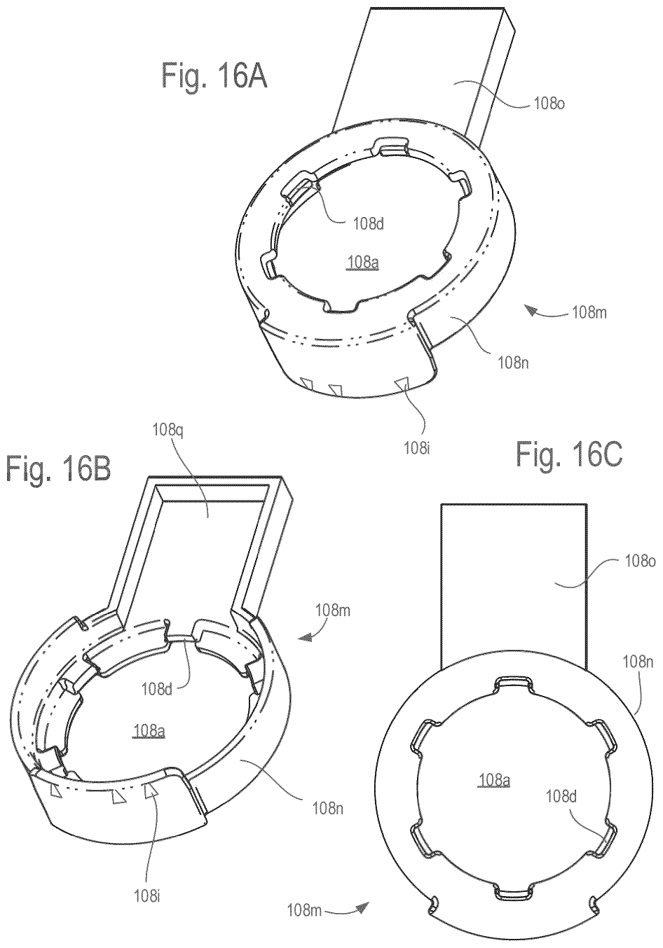

FIGS. 15A-16C show an embodiment of a port housing 108. This embodiment is similar to the embodiment of FIGS. 5A-5D, but includes a connector housing 108m and other features to facilitate connection of a light 120 to a power source or a light guide to a light source. In this case, the port housing 108 includes a slot 108k extending from one or more of the first pivot recesses 108b, preferably in a generally radial direction. When the port housing 108 is assembled with the lock ring 110, the slots 108k each provide access for a respective light 120, such as an electrical wire or a fiber optic cable.

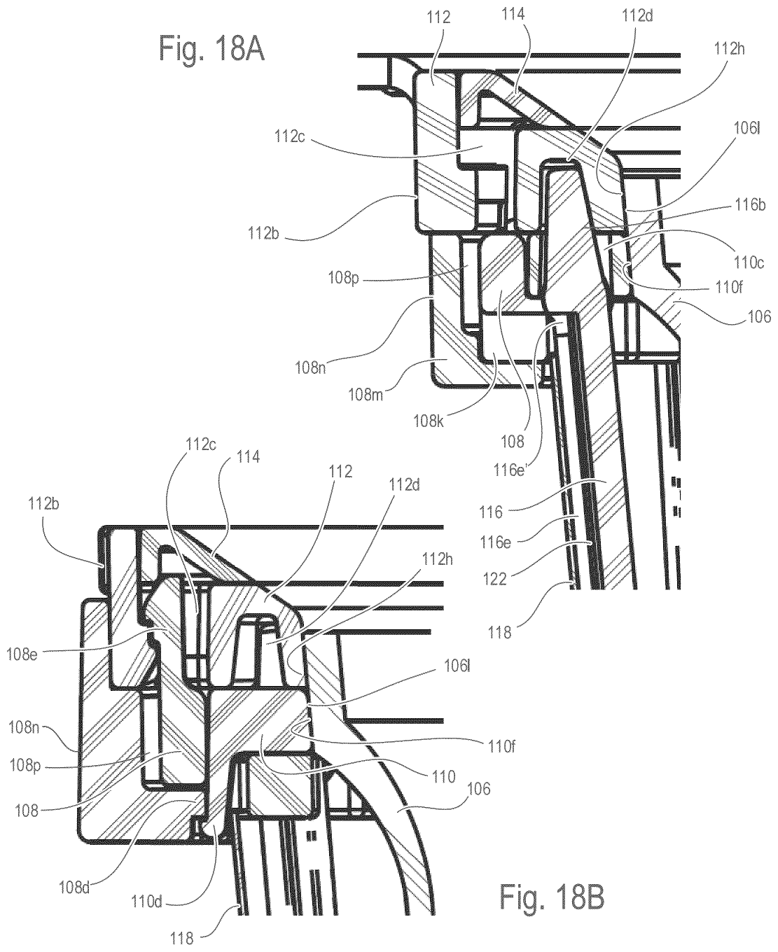

The connector housing 108m is secured to the remainder of the activation arm mounting body to a form a housing interior space 108p (see FIG. 18A) that receives the light connector 122. More specifically, the connector housing 108m has a connector housing outer body 108n that contains the portion of the port housing 108 through which the slots 108k extend. The connector housing outer body 108n also has a connector housing opening 108q through which the light connector 122 passes to connect to a power supply or light supply. The connector housing opening 108q may be located at any suitable location. In this case, the connector housing opening 108q is provided on a connector housing extension 1080 that extends radially from the remainder of the port housing 108. The connector housing extension 108o optionally may be positioned under the extensions 108f to provide relatively little obstruction to the surgical theater.

In this embodiment, the locking tab receivers 108d are provided on the connector housing 108m, such that the locking tab 110d of the lock ring 110 connects to the connector housing 108m with the main body of the port housing 108 captured in place between the lock ring 110 and the connector housing 108m. The locking tab receivers 108d may be recessed towards the lock ring 110, and configured to fit into matching tab receiver recess 1081 formed in the bottom of the main body of the port housing 108, to thereby reduce the overall height of the assembled port housing 108.

The activation arm mounting body also includes one or more body position indicators 108i. The body position indicators 108i are visual, and optionally also tactile, indicators to show the rotational position of the activation ring 112 relative to the port housing 108. As shown in FIGS. 17A-17B, the activation ring 112 likewise has one or more ring position indicators 112g, which align with the body position indicators 108i in different ways depending on the rotational position of the activation ring 112 relative to the port housing 108. In this case, the activation ring 112 has a single ring position indicator 112g, and the port housing 108 has three body position indicators 108i arranged at different circumferential locations. The body position indicators 108i are, in this case, provided on the connector housing 108m, but alternatively may be provided on the main body of the port housing 108 or elsewhere on the activation arm mounting body.

When the activation assembly 102 is assembled, the ring position indicator 112g is positioned adjacent to a first body position indicator 108i′ when the activation ring 112 is in the first position (i.e., when the expandable port 104 is contracted), and a second body position indicator 108i″ when the activation ring 112 is in the second position (i.e., when the expandable port 104 is expanded). The ring position indicator 112g aligns with a third body position indicator 108i″, which is located between the other two body position indicators 108i′, 108i″, when the activation ring 112 is in an intermediate position. This third body position indicator 108i″ may be helpful when the surgeon does not wish to fully expand the expandable port 104. More or fewer body position indicators 108i may be used in other embodiments.

The intermediate position beneficially may be the position at which the distal arm ends 116d and the membrane lip 118c (if present) are just radially outside the annular recess 106f in the introducer 106. Stated differently, the intermediate position may be the position at which the distal arm ends 116d and membrane lip 118c (if present) are spaced radially by the maximum diameter IDI of the introducer tip 106e to thereby allow the introducer 106 to be freely removed in the proximal direction without interfering with the arms 116 and other portions of the expandable structure. This allows the surgeon to open the arms 116 by the minimum amount necessary to remove the introducer 106, as may be helpful in some circumstances.

FIGS. 17A-17B also show the activation ring 112 having knurling on the grip 112b to enhance the feel and usability of the activation ring 112. The knurling may be replaced by other structures to enhance grip, such as a rubber or other high-friction ring, or the like. The activation ring 112 also (or alternatively) may include one or more handles or levers (not shown) for operating the activation ring 112. The activation ring 112 also includes a ring position indicator 112g.

FIGS. 18A-18B are cross-sectional views that show the assembly of the various parts in detail. Here, it can be seen that the lock ring 110 connects via locking tabs 110d to the locking tab receivers 108d to secure the connector housing 108m to the rest of the port housing 108, and capture the main body of the port housing 108 in place. Also, FIG. 18A shows the housing interior space 108p formed between the connector housing 108m and the rest of the port housing 108, and how the slot 108k aligns with the proximal slot end 116e′ of the associated activation arm 116 to provide a passage for the light connector 122. The connection of the port housing 108 to the activation ring 112 via the sliding tabs 108e is also shown. These figures also show how the cam followers 116b extend through the cam follower ports 110c to engage the cam slots 112d, the interaction between the tapered activation ring inner face 112h, introducer outer face 106l and lock ring inner face 110f, and other features.

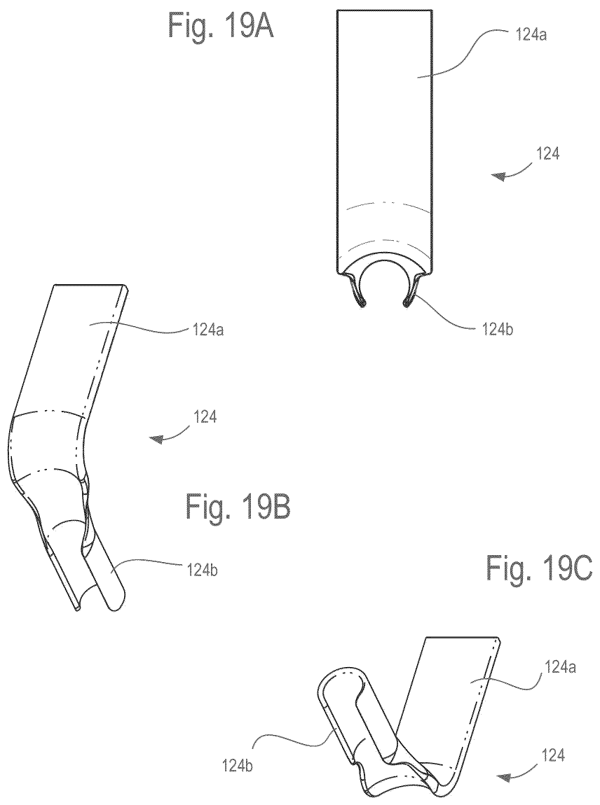

FIGS. 19A-19C show an example of a surgical tool mount 124 that may be used with embodiments of an expandable access port 100. The surgical tool mount 124 includes a tab 124a that is configured to secure to an extension 108f, and a connector 124b that is configured to secure to or guide a surgical instrument, such as an endoscope, a suction hose, a light, and so on. In this case, the connector 124b comprises a cylindrical clip having an open side to allow the clip to flex and generate a restoring force to hold the instrument in place. Other embodiments may have different constructions, as will be apparent to persons of ordinary skill in the art in view of the present disclosure.

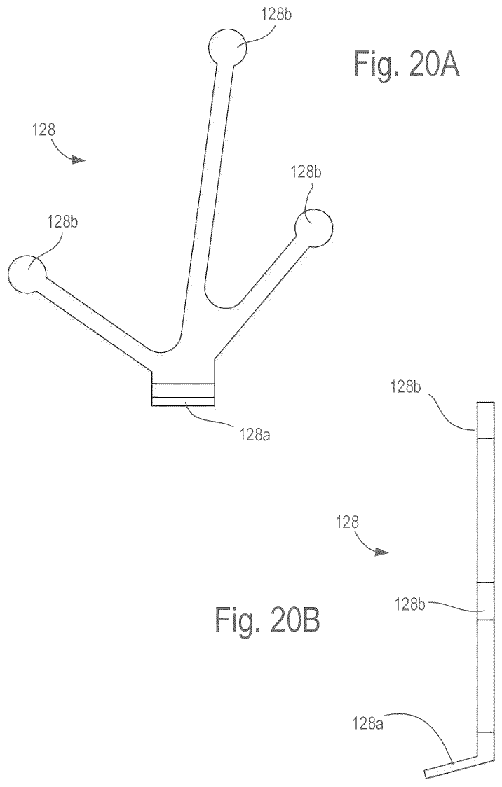

FIGS. 20A-20B show an example of a guidance arm 128 that may be used with embodiments of an expandable access port 100. The guidance arm 128 includes a tab 128a that is configured to attach to the expandable access port 100, such as by securing it to an extension 108f, and an array of indicators 128b, such as reflective spheres or discs, that are used for visually tracking the position of the guidance arm 128 via a stereotactic navigation system, as known in the art. The indicators 128b preferably are mounted to extend from the tab 128a at an angle extending away from the longitudinal axis 100a, to help clear the area above the expandable access port 100 for surgical operations. Similar indicators 128b may also be used in conjunction with a navigation probe 126, as also known in the art. In addition, the indicators 128b may be directly integrated into the body of the expandable access port 100, such as by mounting them on an extension 108f or multiple extensions 108f. Other alternatives and embodiments will be apparent to persons of ordinary skill in the art in view of the present disclosure.

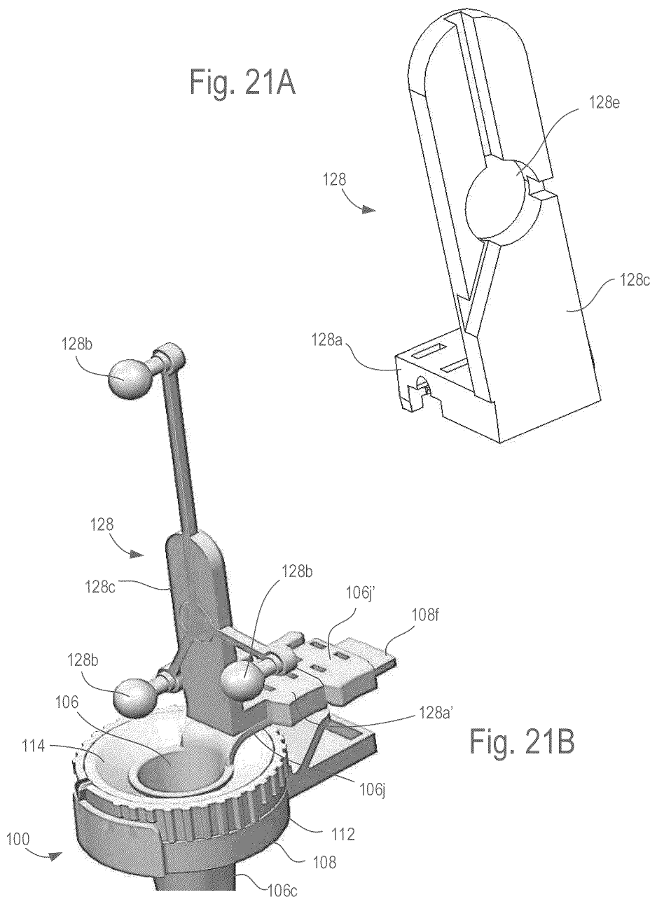

FIGS. 21A-21B show another embodiment of a guidance arm 128. In this case, the guidance arm 128 also includes a tab 128a for connecting to the expandable access port 100 (e.g., by securing to an extension 108f), and an array of indicators 128b. Here, the guidance arm 128 is provided as a two-part assembly with a base 128c to which the indicators 128b are attached. In this example, the indicators 128b are mounted on a frame 128d that fits into a corresponding opening 128e of the base 128c. Magnets or other fasteners may be used to selectively connect the frame 128d to the base 128c. This arrangement allows different arrays of indicators 128b to be used. For example, different frames 128d may be provided with indicators 128b at different locations corresponding to space requirements or particular requirements of different tracking systems.

FIG. 21B also shows how a guidance arm 128 can be mounted to the remainder of the expandable access port 100. In this case, the guidance arm tab 128a is secured by a lock 128a′ to an introducer mounting tab 106j. The introducer mounting tab 106j is secured to a port housing extension 108f by its own lock 106j′. Thus, the guidance arm 128 may be removed separately, or in conjunction with the introducer 106.

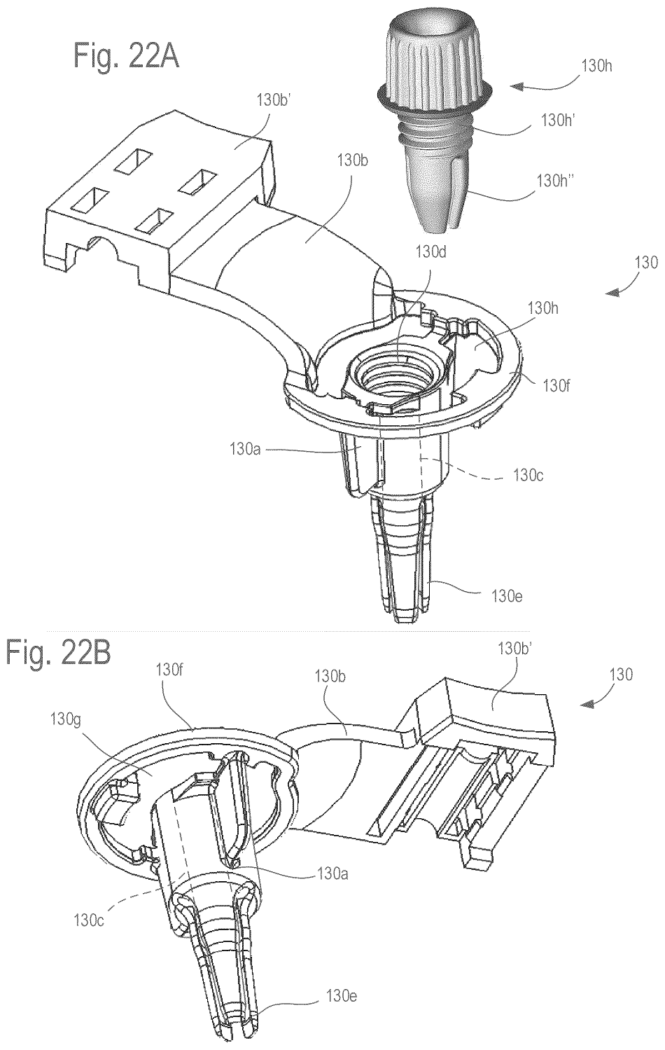

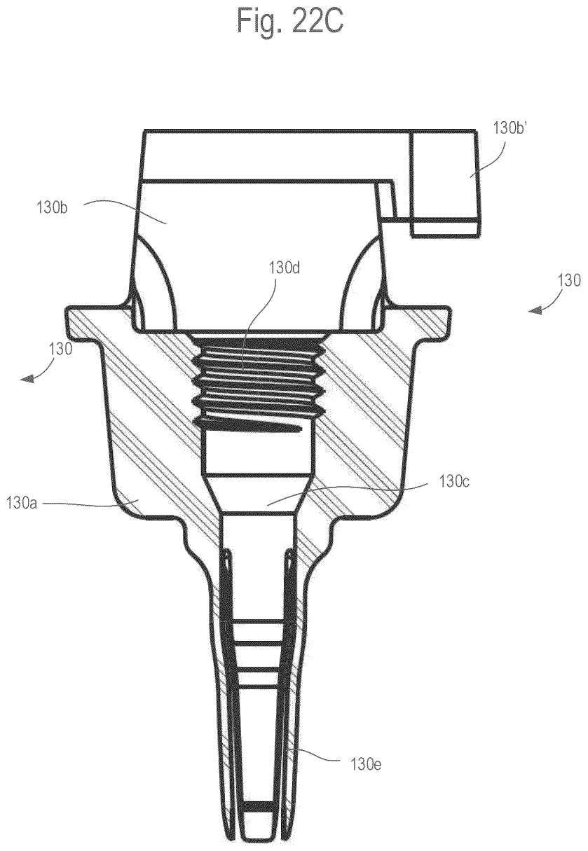

FIGS. 22A-22C show an example of a probe lock 130 that may be used to hold a navigation probe 126 in registration with the expandable access port 100. The probe lock 130 comprises a lock body 130a that extends into the introducer 106, and a tab 130b that is connectable to a port housing extension 108f. the tab 130b includes or is configured to secure to any suitable lock 130b′. The lock body 130a comprises a central passage 130c having a threaded bore 130d at its proximal end, and one or more flexible arms 130e at its distal end. The threaded bore 130d receives external threads 130h′ or a lock nut 130h. The lock nut 130h has a central bore to receive the probe shaft, and tapered fingers 130h″. The tapered fingers 130h″ are compressed radially inward by the inner walls of the central passage 130c as the nut 130h is threaded into the bore 130d, to thereby clamp the navigation probe 126 in place. The flexible arms 130e help to allow navigation probes with different diameters to be used in the probe lock 130 by flexing to fit the particular probe's diameters. When the parts are assembled, the central passage 130c may be collinear with the longitudinal axis 100a, but this is not strictly required. The lock body 130a may be connected to the tab 130b by a ring body 130f having openings 130g through which the surgeon can view down the introducer. Examples of various suitable probe lock features are provided in U.S. application Ser. No. 17/473,282 (publication no. 2021/0401457), which is incorporated by reference herein.

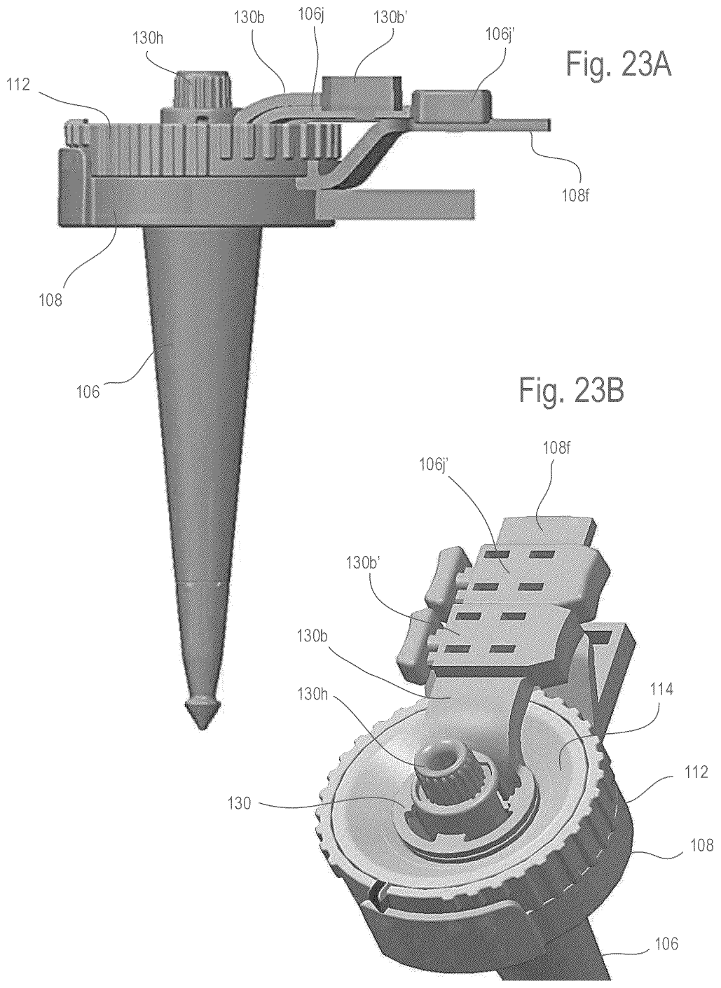

FIGS. 23A and 23B show the probe lock 130 of FIGS. 22A-22C installed with an expandable access port. In this case, the arms 116 and membrane 118 are removed to see the shape of this alternative introducer 106, which is conical. The conical introducer 106 is dimensioned to fit closely within the space formed by the arms 116 when the arms 116 are in their collapsed position, and thereby help support the arms 116 and prevent unwanted flexure as the access port is move to the surgery site. As shown in the figures, the lock tab 130b may be mounted to the remainder of the assembly by connecting its lock 130b′ to the tab 106j of the introducer 106. The introducer tab 106j is, in turn attached by a lock 106j′ to one of the port housing extensions 108f. Other embodiments may connect the parts in other ways.

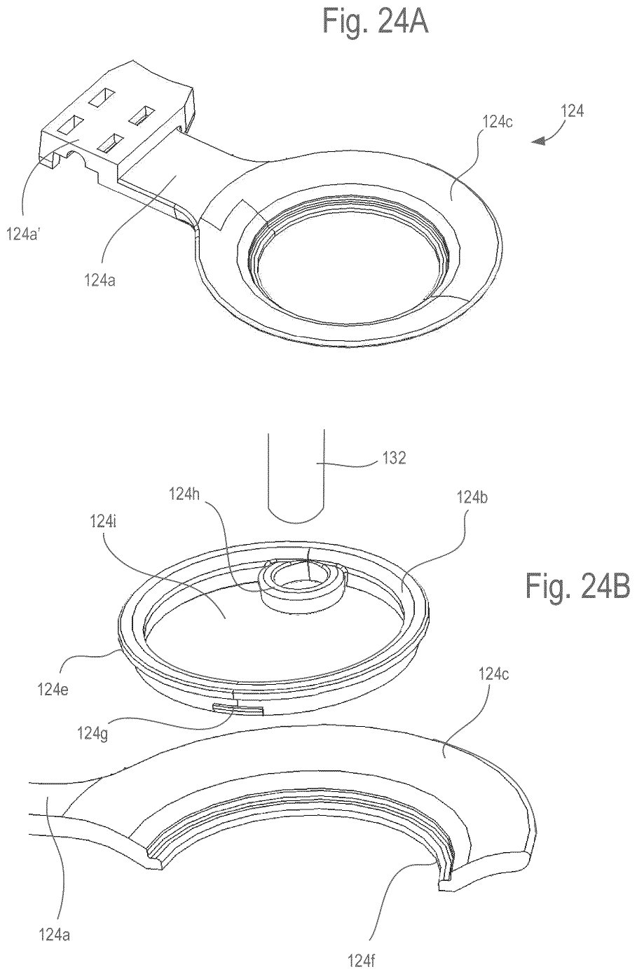

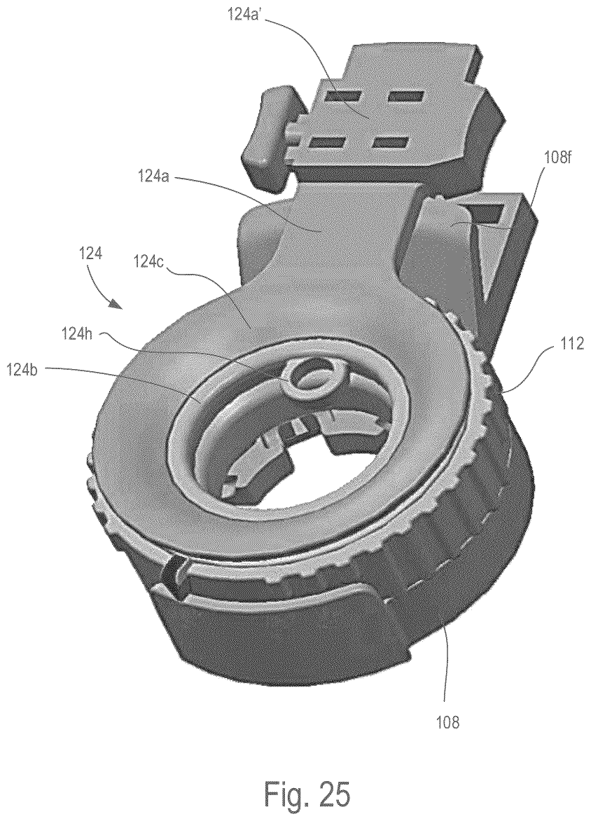

FIGS. 24A and 24B show another example of a surgical tool mount 124. In this case, the surgical tool mount 124 comprises a tab 124a that is configured to secure via a lock 124a′ to a port housing extension 108f, and a ring-shaped mount body 124c. The mount body 124c has a circular central opening 124d that surrounds the activation assembly opening 102a, and a connector 124b that fits within the central opening 124d. The connector 124b preferably is configured to rotate within the central opening 124d. For example, the connector 124b may have an outer rim 124e that fits on top of an inner rim 124f formed in the central opening 124d, and tabs 124g that surround the bottom of the inner rim 124f. The outer rim 124e and tabs 124g form an annular space that captures the inner rim 124f and prevents the connector 124b from separating from the mount body 124c, while still allowing the connector 124b to rotate within the mount body 124c. A ring-shaped connector 124b such as shown is expected to provide smooth rotation by the mating circular surfaces, and allows the surgeon to position the endoscope 132 or other device at the most convenient location. In addition, the connector 124b may be made without any kind of locking device, so that it is freely movable at all times as the need might arise during surgery. Travel stops may be provided, however, to limit rotation to a specific range. Friction between the connector 124b and mount body 124c can hold the connector 124b in a fixed location until the surgeon applies a force to rotate the connector 124b. While free movability is desired in some embodiments, in other cases, a lock, such as a thumb screw, may be provided to hold the connector 124b at a fixed location.

The connector 124b may include any suitable mechanism for holding any one or more types of surgical instrument. For example, the connector 124b may have a tool connector in the form of an inner ring 124h that is dimensioned to hold an endoscope 132. In this case, the inner ring 124h is located at the outer radial edge of the connector 124b, so that the instrument, when installed, is offset from the longitudinal axis 100a. For example, in the shown embodiment, the inner ring 124h is radially offset from the central axis of the opening 124i. The remainder of the connector 124b has an opening 124i that is located within the central opening 124d of the mount body 124c and preferably surrounds the longitudinal axis 100a. In this way, the instrument can be positioned at any desired angular location about the longitudinal axis 100a, and still allow access for other instruments to be used within the expandable port 100.

The inner ring 124h (or other types of tool connector) may use any suitable lock or holding mechanism to hold the instrument. For example, the inner ring 124h may comprise a circular opening that is dimensioned to snugly fit the outer surface of the endoscope 132 such that the endoscope 132 can be moved distally and proximally by the surgeon by hand, while still holding the endoscope in any position when the surgeon releases the endoscope 132. The inner ring 124h also may have a radial slot such that the inner ring 124h is defined by two arms, in which case the arms may be flexible to provide a resilient force to grip a surgical instrument positioned between the arms. A separate lock device, such as a locking screw, may also be used. Materials, such an overmolded high-friction elastomer, may be used to modify the operation of the inner ring 124h. Other embodiments may use clamps compression nut arrangements (e.g., like threaded bore 130d and nut 130h) or the like. Other alternatives and embodiments will be apparent to persons of ordinary skill in the art in view of the present disclosure.

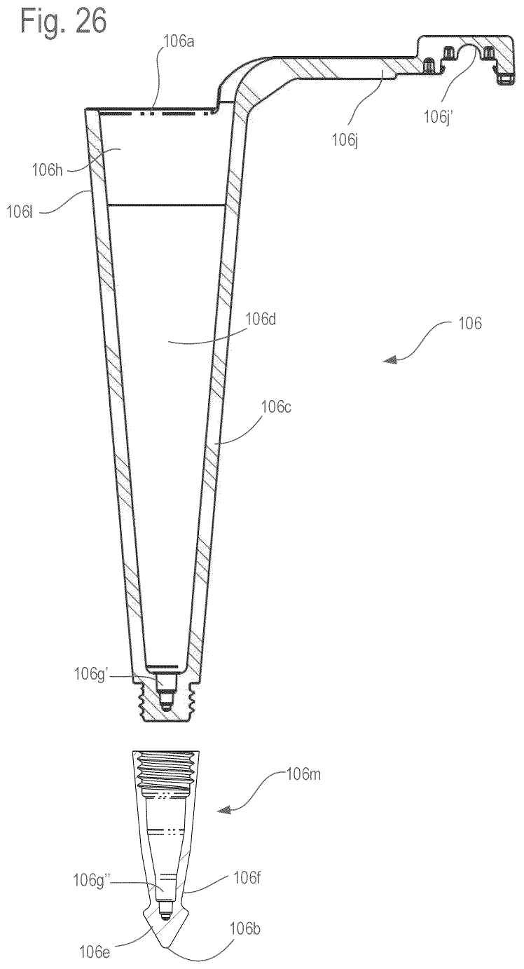

FIG. 26 illustrates the exemplary introducer 106 of FIGS. 23A and 23B in cross-section to show additional optional details. The introducer 106 is similar to the one in FIGS. 11A-11C, but has an integral lock 106j′, a continuous conical tapered wall 106c forming the cannula 106d, and a separately formed tip unit 106m. The tip unit 106m includes the introducer tip 106e, and optionally an annular recess 106f. The tip unit 106m is configured to permanently or removable attach to the rest of the introducer 106. In the shown example, the tip unit 106m has internal threads 106n that engage external threads 1060 on the tapered wall 106c. In other cases, snap fitments may be used (e.g., a non-reversible snap connector), or the tip unit 106m may be secured by adhesives, welding, and so on.

The tip unit 106m is provided to allow the use of different navigation devices with different probe shaft lengths. For example, in the shown embodiment, the introducer cannula 106d terminates to form a probe tip receiver 106g′ at the end of the conical wall 106c. In use, the probe shaft tip seats in the probe tip receiver 106g′, and is offset from the distal introducer end 106b by a fixed, known distance. This distance can be used to offset the calibration of the navigation system. Thus, in those cases in which a probe shaft is not long enough to extend to the distal introducer end 106b, the two-part introducer shown of FIG. 26 can be used to account for the shorter length.

The introducer 106 of FIG. 26 can also be modified in various ways. For example, the introducer cannula 106d may terminate at a simple opening that allows the navigation probe shaft to pass through to be seated in a probe tip receiver 106g″ in the tip unit 106m. It is also envisioned that introducers 106 having various constructions may be provided as a kit for use in different configurations as required by the particular circumstances. For example, an introducer tip unit 106m may be provided with one or more introducer bodies as shown (i.e., having a probe tip receiver 106g′), along with one or more introducer bodies that has have openings to allow the probe shaft pass into the tip unit 106m. This may be helpful, for example, to allow the surgeon to select between different introducer lengths and configurations for the particular case, and then secure the tip to the desired introducer body for use in surgery. Other alternatives and embodiments will be apparent to persons of ordinary skill in the art in view of the present disclosure.

Having explained details of embodiments of expandable access ports 100, the operation will be understood. For example, the expandable access port 100 may be operated by connecting the introducer 106 to the activation assembly 102 with the introducer tip 106e positioned distally beyond the distal arm ends 116d, rotating the activation ring 112 to move the activation arms 116 to the first (contracted) positions, then inserting the assembled expandable access port 100 to the surgery site, operating the activation ring 112 to move the activation arms 116 to the second (expanded) positions, removing the introducer 106, and performing a surgical procedure through the expanded 104. Additional optional steps include, but are not limited to, installing a navigation probe 126 in the introducer 106 or attaching a guidance arm 128 to the activation assembly 102 and using the navigation probe 126 or guidance arm 128 to guide the expandable access port 100 into place using stereotactic navigation, as known in the art. Also, a navigation probe 126 may be attached to the introducer 106 to guide the expandable access port 100 during initial insertion, and a guidance arm 128 may be used to ensure continued placement while the expandable access port 100 is used for the surgical procedure or to reposition the expandable access port 100 during surgery. Other optional steps include operating a light 120 to illuminate the surgery site, and mounting a surgical tool mount 124 to the activation assembly 102 to hold or guide additional surgical instruments, and the like.

It will also be understood that method of manufacturing an expandable access port 100 are also encompassed by this disclosure. For example, an expandable access port 100 may be manufactured by: providing an activation assembly 102 defining an activation assembly opening 102a surrounding a longitudinal axis 100a; providing a plurality of activation arms 116 arranged around the longitudinal axis 100a, each activation arm 116 in a distal direction D from a respective proximal arm end 116g to a respective distal arm end 116d, with each respective distal arm end 116d being movably connected to the activation assembly 102 and movable, upon operation of the activation assembly 102, between a respective first position in which each respective distal arm end 116d is spaced a respective first distance D1 from the longitudinal axis 100a, and a respective second position in which each respective distal arm end 116d is spaced a respective second distance D1 from the longitudinal axis 100a, wherein each respective second distance D2 is greater in magnitude than each respective first distance D1; and overmolding a membrane 118 onto the plurality of activation arms 116, the membrane 118 extending in the distal direction D from a proximal membrane end 118a adjacent the activation assembly 102 to a distal membrane end 118b adjacent the respective distal arm ends 116d, wherein the membrane 118 comprises a flexible material that is expandable to permit the plurality of activation arms 116 to move from the respective first positions to the respective second positions. Optional steps to this method include, but are not limited to, forming the membrane 118 from materials and with dimensions and other properties as described above, overmolding the membrane 118 into slots 116e formed on the activation arms 116, capturing a light 120 in place within a slot 116e, overmolding the membrane 118 into a structure comprising ribs 118d on the outer radial surface 116h of each activation arm 116 and walls 118e between each adjacent pair of ribs 118d, and overmolding a lip 118c around the distal arm ends 116d.

The various parts of the expandable access port may be made of any material that is suitable for the purposes herein, and has adequate biocompatibility for use in a surgical setting. For example, the activation arms 116 and membrane 118 of the expandable port 104 may be constructed as described above, and may be transparent or semi-transparent to allow visualization of the brain tissue surrounding the expandable port 104. Similarly, the introducer 106, port housing 108, lock ring 110, activation ring 112 and activation ring cover 114 may be constructed of polycarbonate or other materials, and may be transparent or opaque.

The foregoing embodiments are expected to provide enhanced brain access performance, such as in the form of ease of use, providing a suitable working space, accuracy in placing the device at the desired location, mitigating trauma to the surrounding tissue, and cost-effectiveness. However, the inventors have determined that additional modifications can enhance performance of the device in relation to providing a functionally-enlarged surgical working space without unnecessarily increasing trauma to the brain tissue. In particular, it has been determined that embodiments can be configured to provide an elongated working channel—that is, a working channel having a relatively large major axis, and a relatively small minor access perpendicular to the major axis. The exact shape of the elongated working channel can vary among embodiments.

Elongation of the working channel is expected to provide various benefits. For example, the larger major axis can enhance binocular vision to the surgery site and improve bimanual use of tools or the simultaneous use of multiple tools within the access port, while the smaller minor axis does not apply unnecessary pressure on adjacent portions of the brain tissue. Thus, greater accessibility and usability are provided, while reducing overall trauma on the tissue.

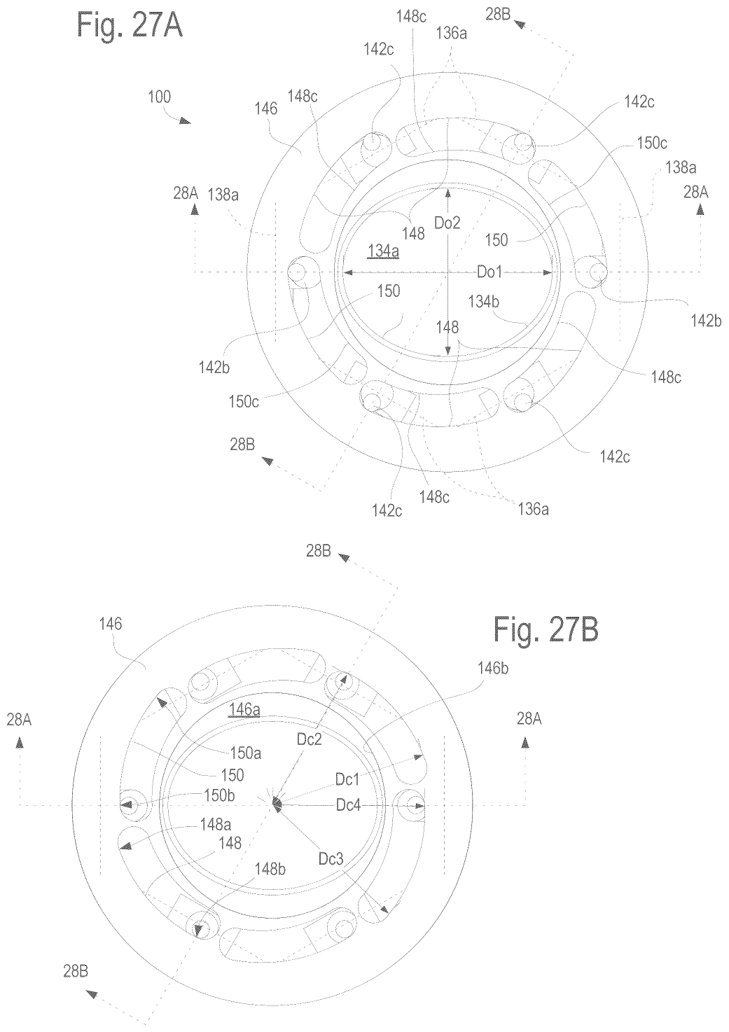

FIGS. 27A-43C provide additional embodiments of expandable access ports 100 that are configured to provide an elongated working channel having a relatively large major axis and a relatively small minor axis during insertion (i.e., in the closed position) and preferably also during operation (i.e., in the expanded or opened position). For clarity of illustration, only certain features are shown and/or identified with a reference number in each Figure. It will be understood that other features as shown in the preceding embodiments may be integrated into the subsequently described and illustrated embodiments. For example, details of the activation assembly are not shown in these drawings, but may be included according to the preceding portions of the disclosure.

Referring now to FIGS. 27A-29B, a first exemplary embodiment of an expandable access port 100 is shown in the closed position (FIGS. 27A, 28A, 29A), and the opened position (FIGS. 27B, 28B, 29B). The access port 100 comprises an arm mounting body 134 defining an arm mounting body opening 134a, a number of primary pivot locations 136, and a number of secondary pivot locations 138. The arm mounting body 134, arm mounting body opening 134a, primary pivot locations 136, and secondary pivot locations 138 may be formed as described above in relation to the assembly of the port housing 108 and lock ring 110, or according to other constructions. The arm mounting body opening 134a, primary pivot locations 136, and secondary pivot locations 138 surround a longitudinal axis 140.

The arm mounting body opening 134a, as defined by a perimeter wall 134b, has an elongated cross-section as viewed along the longitudinal axis 140. More specifically, the cross-section extends along a major opening axis, and along a minor opening axis that is perpendicular to the major opening axis. As used generally herein, the “major” axis is the axis in which a cross-section of the shape, as viewed along the longitudinal axis 140, has a larger dimension, and the “minor” axis is the axis in which the cross-section of the shape, as viewed along the longitudinal axis 140, has a smaller dimension. Thus, by this definition, the measured dimension of cross-section is greater along the major axis than along the minor axis. Thus, in this case, the major opening axis dimension Do1 is greater than the minor opening axis dimension Do2. In the shown embodiment, the arm mounting body opening 134a is ovate (i.e., elongated and having rounded perimeter walls in the shape of an oval, ellipse, or the like). In other embodiments, the arm mounting body opening 134a may have one or more linear walls, and may have angled corners (e.g., in the shape of a rectangle). The elongated arm mounting body opening 134a facilitates binocular vision and bi-manual operation of instruments in the working channel, and allows the dimension of the membrane 118 to be minimized in the minor opening axis direction.

A number of primary arms 142 are arranged around the longitudinal axis 140. Each primary arm 142 is pivotally attached to the arm mounting body 134 at a respective primary pivot location 136 via a pivot 142a. Each primary arm 142 extends, in a distal direction D along the longitudinal axis 140, to a respective primary arm distal end 142b. Each primary arm 142 also includes a respective primary cam follower 142c, which may be defined on either side of the pivot 142a (i.e., with the cam follower 142c between the pivot 142a and the distal end 142b, or the pivot 142a between the cam follower 142c and the distal end 142b). The primary cam follower 142c may have any suitable shape to perform the function as described below.

Each primary pivot 136 location defines a respective primary pivot axis 136a about which the respective primary arm 142 rotates. Each primary pivot axis 136a preferably extends in a plane that is perpendicular to the longitudinal axis 140, and one or all of the primary pivot axes 136a preferably may be oriented to be tangential to the longitudinal axis 140.

A number of secondary arms 144 are arranged around the longitudinal axis 140. Each secondary arm 144 is pivotally attached to the arm mounting body 134 at a respective secondary pivot location 138 via a pivot 144a. Each secondary arm 144 extends, in a distal direction D along the longitudinal axis 140, to a respective secondary arm distal end 144b. Each secondary arm 144 also includes a respective secondary cam follower 144c, which may be defined on either side of the pivot 144a (i.e., with the cam follower 144c between the pivot 144a and the distal end 144b, or the pivot 144a between the cam follower 144c and the distal end 144b). The secondary cam follower 144c may have any suitable shape to perform the function as described below.

Each secondary pivot 138 location defines a respective secondary pivot axis 138a about which the respective secondary arm 144 rotates. Each secondary pivot axis 138a preferably extends in a plane that is perpendicular to the longitudinal axis 140, and one or all of the secondary pivot axes 138a preferably may be oriented to be tangential to the longitudinal axis 140.

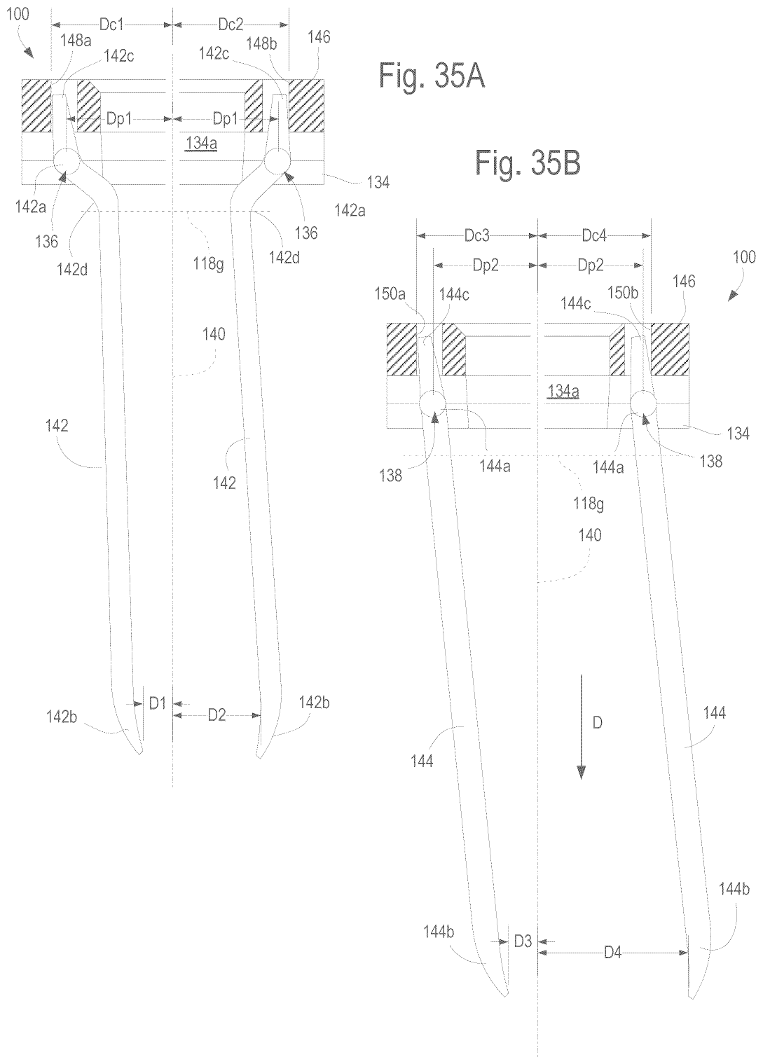

The primary arms 142 and secondary arms 144 may be constructed in general principle according to the embodiments described above, and may include features such as slots and light receivers, inward bends at the ends or along the middle (see FIG. 35A), and so on. An expandable membrane 118 is attached directly or indirectly to one or all of the primary arms 142 and secondary arms 144, and may be directly or indirectly connected to the arm mounting body 134, in a manner comparable to the embodiments described above.

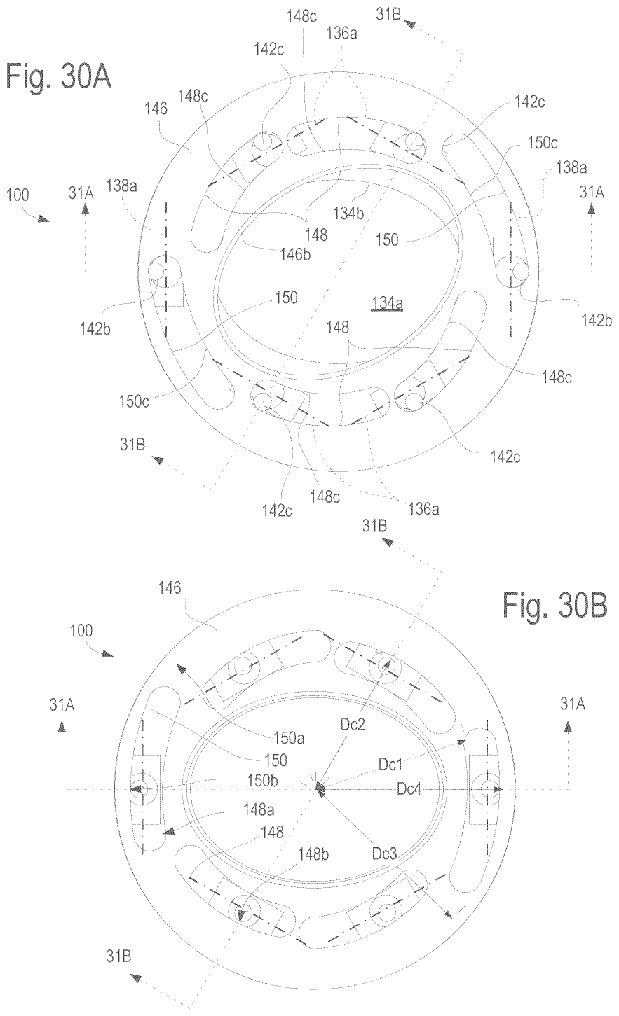

The access port 100 also includes an activation ring 146 that is rotatably mounted to the arm mounting body 134 to move between a first activation ring position corresponding to the closed access port position (FIGS. 27A, 28A, 29A), and a second activation ring position corresponding to the opened access port position (FIGS. 27B, 28B, 29B). The activation ring 146 may be constructed in general principle according to the preceding embodiments, and may, for example, be constructed with knurling or other grip features, position indicators, and the like. The activation ring 146 has an activation ring opening 146a, defined by a perimeter all 146b, that is aligned with the arm mounting body opening 134a. In this case, the activation ring opening 146a is circular, but it alternatively might be elongated (see, e.g., FIG. 30A).

In one preferred embodiment, the activation ring 146 may be constructed (such as described above) so that the activation ring 146 remains at a fixed location along the longitudinal axis 140, relative to the arm mounting body 134, throughout rotation of the activation ring 146 between the first activation ring position and the second activation ring position. This prevents “telescoping” of the activation ring 146 and the remainder of the expandable access port 100, which can complicate the process of placing and maintaining the position of the access port 100 during surgery.

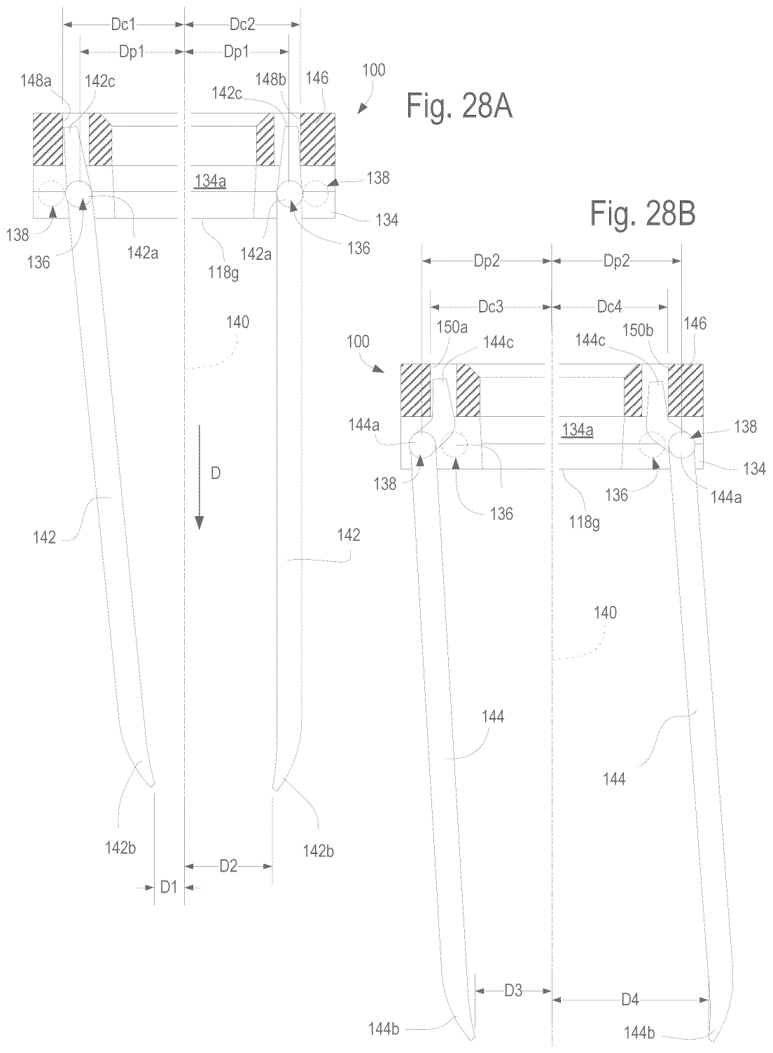

The activation ring 146 has a number of primary cam surfaces 148, each of which is operative to engage a respective primary arm 142 and generate a force to drive the primary arm 142 from a closed position to an opened position. FIG. 28A is a composite view showing a cross-section as viewed along line 28A-28A in FIG. 27A (closed position) on the left, and a cross-section as viewed along line 28A-28A in FIG. 27B (opened position) on the right. Thus, to be clear, FIG. 28A shows two different operative positions (one on each side of the figure), rather than a single operating state of the device. The same idea is true for other composite views described herein. In the closed position the primary arm distal end 142b is located a first distance D1 from the longitudinal axis 140, and in the opened position the primary arm distal end 142b is located a second distance D2 from the longitudinal axis 140. The second distance D2 is greater than the first distance D1.

Each primary cam surface 148 may extend about the longitudinal axis from a respective first cam end 148a at a first cam distance Dc1 from the longitudinal axis 140 to a respective second cam end 148b at a second cam distance Dc2 from the longitudinal axis 140. The second cam distance Dc2 is less than the first cam distance Dc1. The primary cam surface 148 may extend along a linear path, or a curved path. In a preferred embodiment, the path is curved with a concave side facing towards the longitudinal axis 140. The primary cam surface 148 also may comprise a combination of curved and linear paths. For example, a portion of the path may have a continuous uniform distance from the longitudinal axis 140 to provide a range of movement in which the cam surface 148 does not move the primary arm 142 away from the longitudinal axis 140.

Similarly, the activation ring 146 has a number of secondary cam surfaces 150, each of which is operative to engage a respective secondary arm 144 and generate a force to drive the secondary arm 144 from a closed position to an opened position. FIG. 28B is a composite view showing a cross-section as viewed along line 28B-28B in FIG. 27A (closed position) on the left, and a cross-section as viewed along line 28B-28B in FIG. 27B (opened position) on the right. In the closed position the secondary arm distal end 144b is located a third distance D3 from the longitudinal axis 140, and in the opened position the secondary arm distal end 144b is located a fourth distance D4 from the longitudinal axis 140. The fourth distance D4 is larger than the third distance D3.

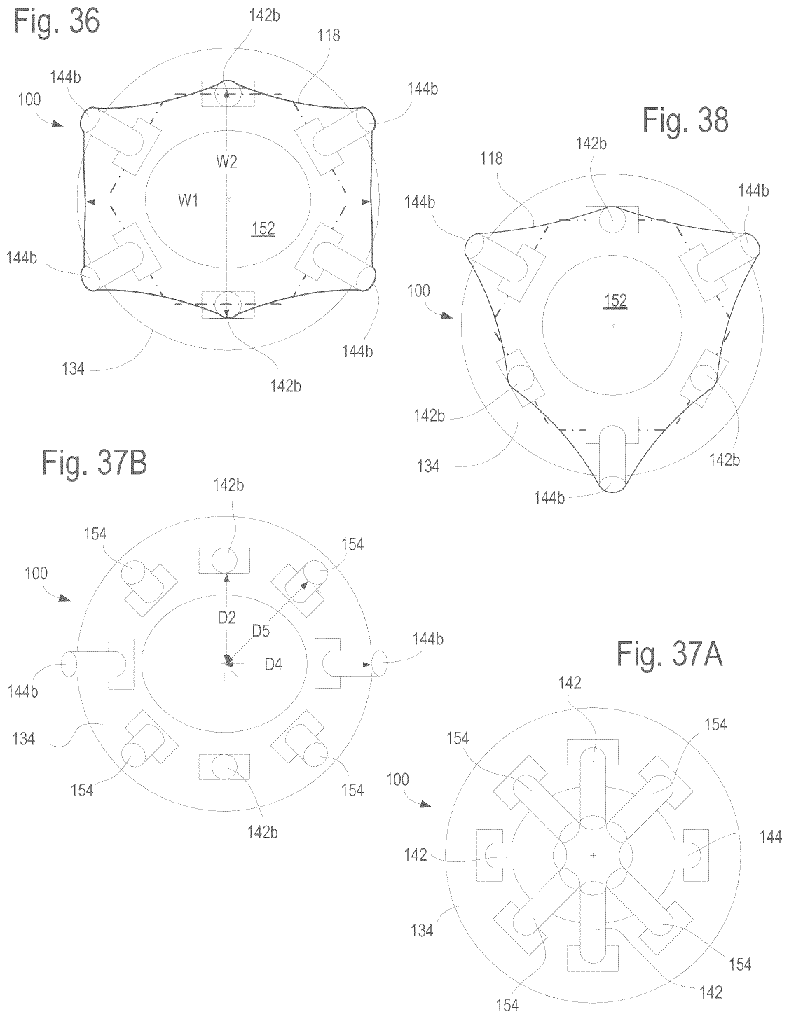

As shown in FIGS. 27A and 27B, the secondary arms 144 may be oriented along the arm mounting body opening major axis dimension Do1, so as to align dimensions D3 and D4 with the major axis of the arm mounting body opening 134a, but this is not strictly required (see, e.g., FIG. 36).

Each secondary cam surface 150 may extend about the longitudinal axis 140 from a respective first cam end 150a at a third cam distance Dc3 from the longitudinal axis 140 to a respective second cam end 150b at a fourth cam distance Dc4 from the longitudinal axis 140. The fourth cam distance Dc4 is less than the third cam distance Dc3. The secondary cam surface 150 may extend along a linear path, or a curved path. In a preferred embodiment, the path is curved with a concave side facing towards the longitudinal axis 140. The secondary cam surface 150 also may comprise a combination of curved and linear paths. For example, a portion of the path may have a continuous uniform distance from the longitudinal axis 140 to provide a range of movement in which the cam surface 150 does not move the secondary arm 144 away from the longitudinal axis 140.

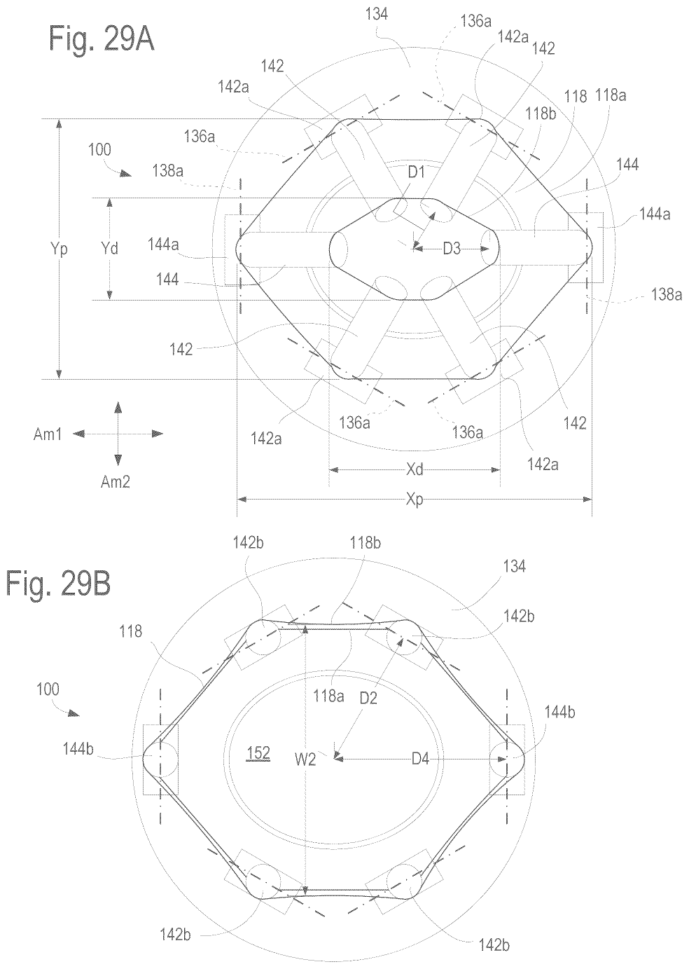

As can be seen in FIGS. 28A-29B, the fourth distance D4 is greater than the second distance D2. Thus, each secondary arm distal end 144b is located further from the longitudinal axis 140 than each primary arm distal end 142a. As a result, the primary arm distal ends 142b and secondary arm distal ends 144b stretch the expandable membrane 118 to define an elongated workspace 152 having a primary axis W1 and a secondary axis W2. For purposes of measuring this dimension the distance across the expandable membrane 118 may be measured, without considering the space occupied by the arms 142, 144 if the arms 142, 144 are present within the boundary of the expandable membrane 118.

The expandable access port 100 also preferably is configured such that at a proximal position (e.g., at or near the arm mounting body 134) the expandable membrane 118 has an elongated membrane cross-section, as viewed along the longitudinal axis 140, having a major membrane axis Am1 and a minor membrane axis Am2. As before, the major membrane axis Am1 is the axis of greater dimension than the minor membrane axis Am2. In the embodiment of FIGS. 27A-29B, the entire expandable membrane 118 has an elongated membrane cross-section from the proximal membrane end 118a at the arm mounting body 134 to the distal membrane end 118b at the primary arm distal ends 142b and the secondary arm distal ends 144b. More specifically, at the proximal membrane end 118a the expandable membrane 118 has a cross-section defined by a larger dimension Xp along the major membrane axis Am1 and a smaller dimension Yp along the minor membrane axis Am2, and at the distal membrane end 118b the expandable membrane 118 has a cross-section defined by a larger dimension Xd along the major membrane axis Am1 and a smaller dimension Yd along the minor membrane axis Am2. Additionally, in this case, the entire expandable membrane 118 has an elongated membrane cross-section in both the closed position and the opened position.

In the embodiment of FIGS. 27A-29B, the desired cross-section of the expandable membrane 118, as well as the shape of the elongated workspace 152 defined by the opened expandable membrane 118, is provided at least in part by the locations of the primary pivot locations 136 and the secondary pivot locations 138. Each primary pivot location 136 is located a first pivot location distance Dp1 from the longitudinal axis 140, and each secondary pivot location 138 is located a second pivot location distance Dp2 from the longitudinal axis 140, with the second pivot location distance Dp2 being greater than the first pivot location distance Dp1. (The dimensions Dp1 and Dp2 may be measured at the rotation axis defined at each pivot location 136, 138.) The primary cam followers 142c and secondary cam followers 144c are located at the same distance from the longitudinal axis 140, but the secondary cam followers 144c are offset towards the longitudinal axis 140 relative to the secondary arm pivots 144a.

The embodiment of FIGS. 27A-29D has primary arms 142 and secondary arms 144 having different constructions, at least with respect to the arrangement between the respective pivots 142a, 144a and cam followers 142c, 144c. However, the activation ring 146 can be constructed with the primary cam surfaces 148 and secondary cam surfaces 150 being identical in shape and dimensions. That is, the first cam distance Dc1 can equal the third cam distance Dc3, and the second cam distance Dc2 can equal the fourth cam distance Dc4. This allows the activation ring 146 to be assembled to the remainder of the device without regard to placing any particular cam surface adjacent to any particular arm (assuming that the arms are equally distributed around the longitudinal axis 140). This also allows the same activation ring 146 part to be used to make an expandable access port 100 having an elongated workspace 152 or, alternatively, an expandable access port 100 that does not provide an elongated workspace (e.g., as in the embodiments described above in relation to FIGS. 1-18B).

If desired, the primary arms 142 and secondary arms 144 also may be configured such that they form an elongated opening when they are in the closed position, as shown in FIG. 29A. In this case, the third distance D3 is greater than the first distance D1. This embodiment can be provided by, for example, selecting the respective shapes of the primary arms 142 and secondary arms 144, and particularly the portion of each arm 142, 144 extending from the pivot 142a, 144a to the distal end 142b, 144b.

Optionally, the arm mounting body 134 may be configured to allow the surgeon or manufacturer to select the particular arrangement of primary arms 142 and secondary arms 144. For example, the arm mounting body 142 may have pairs of primary pivot locations 136 and secondary pivot locations 138 provided around the longitudinal axis 140, so that a primary arm 142 or secondary arm 144 can alternately be located at each position.

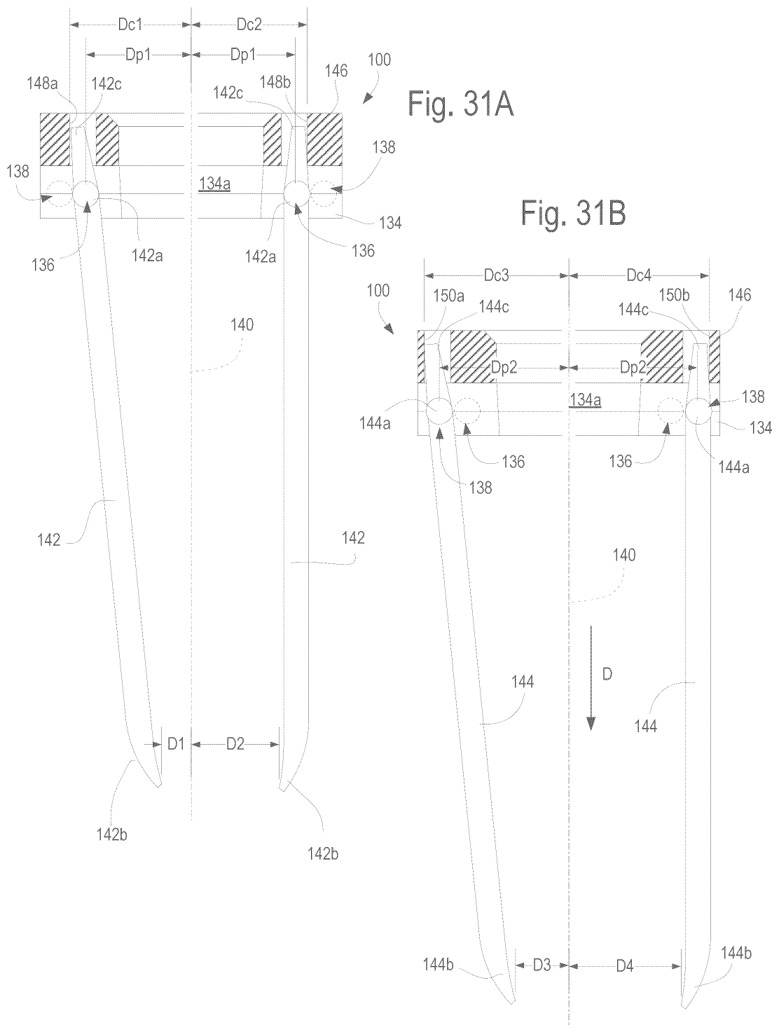

The embodiment of FIGS. 30A-31B provides a different arrangement for achieving an elongated membrane cross-section and an elongated workspace 152. Like in the previous embodiment, the primary pivot locations 136 are placed at a first pivot location distance Dp1 from the longitudinal axis 140, and the secondary pivot locations 138 are placed at a second pivot location distance Dp2 from the longitudinal axis 140, with the second pivot location distance Dp2 being greater than the first pivot location distance Dp1. In this case, the primary arms 142 and secondary arms 144 may be identical in construction, which permits efficiency in constructing the arms 142, 144. The arms 142, 144 also may be identical to activation arms 116 used in expandable access ports 100 that do not provide an elongated workspace, such as the embodiments described above in relation to FIGS. 1-18B.

In this case, the primary cam surfaces 148 and secondary cam surfaces 150 are offset relative to each other with respect to the longitudinal axis 140. The first cam distance Dc1 and second cam distance Dc2 are both less than the third cam distance Dc3 and the fourth cam distance Dc4. The primary and secondary cam surfaces 148, 150 can otherwise be constructed as described above or according to other variations.

In operation, the primary arm distal ends 142b and secondary arm distal ends 144b can begin in an elongated arrangement, as shown in FIG. 29A, and expand into a larger elongated workspace 152, as shown in FIG. 29B. Similarly, the expandable membrane 118 has an elongated membrane cross-section extending from the arm mounting body 134 all the way to the distal arm ends 142b, 144b, in both the opened and closed position.

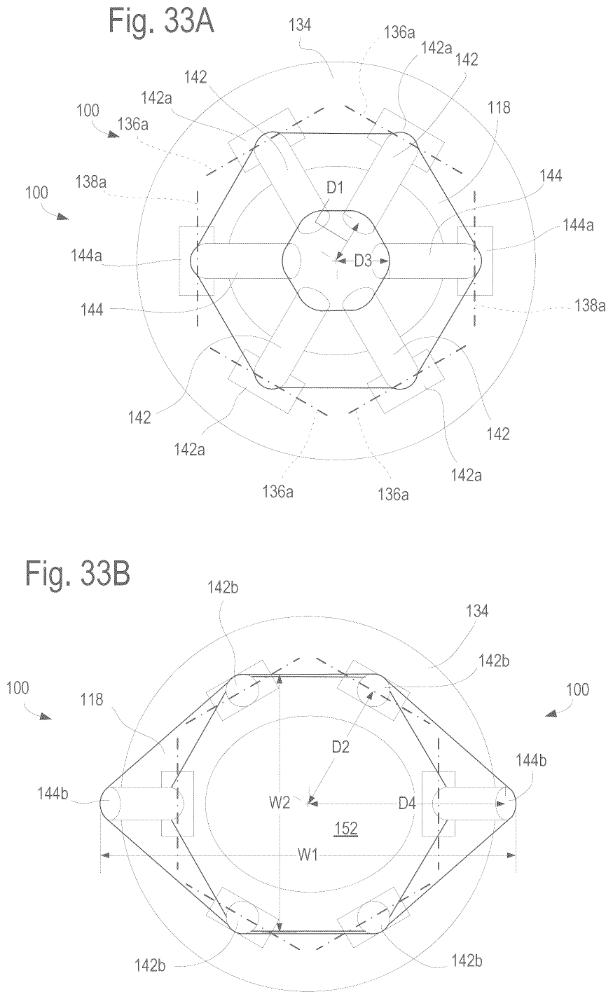

However, embodiments alternatively may be provided in which the distal arm ends 142b, 144b are arranged in a non-elongated closed position (see, e.g., FIG. 33A), in which case the expandable membrane 118 would not have an elongated membrane cross-section at the distal membrane end 118b.

The embodiment of FIGS. 30A-31B also includes an elongated activation ring opening 146a. In this case, the elongated activation ring opening 146a has a major axis that aligns with the major membrane axis Am1 when the activation ring 146 is in the second position (i.e., the opened position) of the arms. This may be beneficial to provide a compact arrangement of parts with a maximized workspace, while still providing sufficient material in the body of the activation ring 146 to form counter-faces 148c as discussed below.

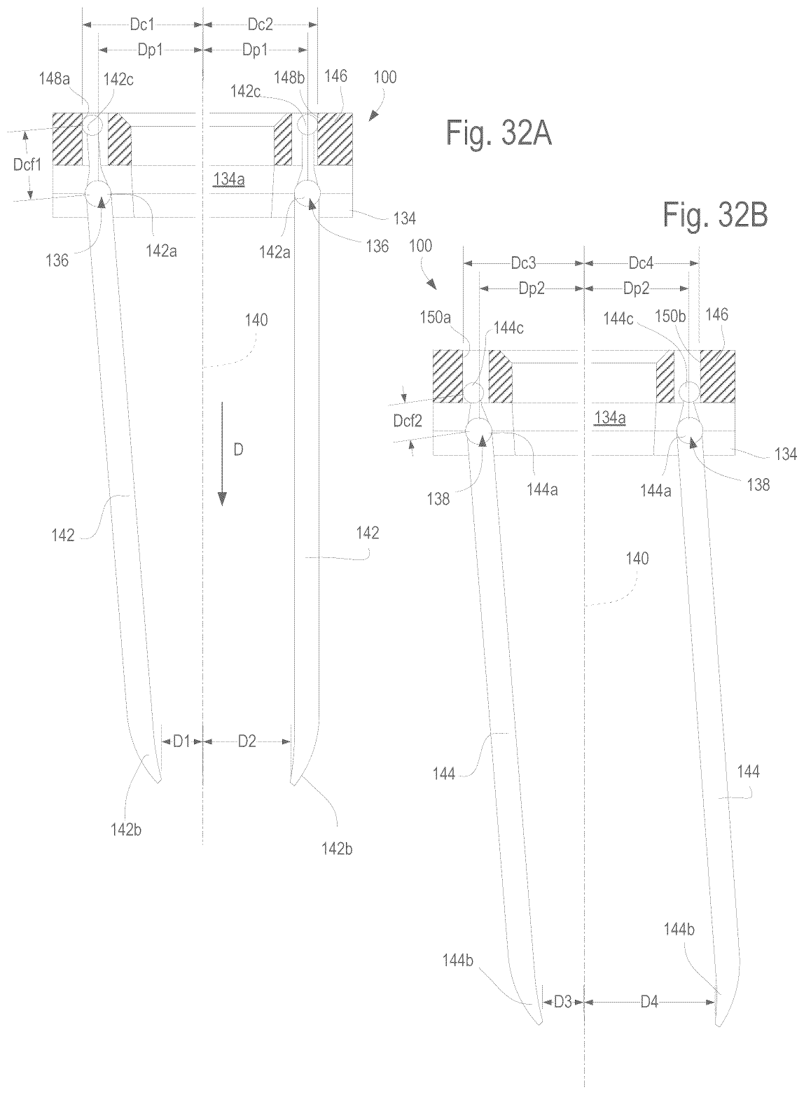

FIGS. 32A-33B show another embodiment of an expandable access port 100 that provides an elongated workspace 152. In this case, the primary pivot locations 136 and secondary pivot locations 138 may be located at the same distance from the longitudinal axis 140 (i.e., the first pivot location distance Dp1 is equal to the second pivot location distance Dp2). In addition the primary cam surfaces 148 and secondary cam surfaces 150 may be identical in shape (e.g., as in the embodiment of FIGS. 27A-29B).

In this example, the expandable membrane 118 may not have an elongated cross-section while in the closed position. However, when opened, the expandable membrane 118 will have an elongated cross-section extending from the distal membrane end 118b to a point proximate to the arm mounting body 134. The desired expansion of the elastic membrane 118 to achieve the elongated cross-section, as well as the elongation of the elongated working space 154, is provided by making the primary arms 142 and secondary arms 144 with different lever ratios. Specifically, each primary cam follower 142c is located a first cam follower distance Dcf1 from the respective primary pivot axis 136a, and each secondary cam follower 144c is located a second cam follower distance Dcf2 from the respective secondary pivot axis 138a. (Dimensions Dcf1 and Dcf2 may be measured as a distance between the respective pivot axis 136a, 138a and an average value, throughout the full range of motion, of the point of contact between each cam follower 142c, 144c and the respective cam surface 148, 150.) The second cam follower distance Dcf2 is less than the first cam follower distance Dcf1. This provides a greater leverage ratio for the secondary arms 144. Thus, a given amount of movement of the primary cam surface 148 towards the longitudinal axis 140 (e.g., 1 millimeter) will rotate the primary arm 142 about a first angle (e.g., 10 degrees), while that same amount of movement (1 millimeter) of the secondary cam surface 150 will rotate the secondary arm 144 about a second angle that is greater than the first angle (e.g., 15 degrees).

The arrangement of FIGS. 32A-33B allows the arm mounting body 132 and activation ring 146 to be constructed, if desired, with the first pivot location distances Dp1 equal to the second pivot location distances Dp2, and the primary cam surfaces 148 identical to the secondary cam surfaces 150. In this case, the differential expansion desired to create the elongated workspace 152 can be achieved solely by using primary arms 142 and secondary arms 144 having different cam follower distances Dcf1, Dcf2. This can provide efficiencies with respect to ease of assembly, and the ability to use activation rings and arm mounting bodies that can also be constructed to provide access ports that do not provide an elongated workspace. For example, the embodiments of FIGS. 1-18B can be modified by replacing some of the activation arms with activation arms having different cam follower distances, in order to convert an expandable access port 100 that provides a circular (non-elongated) workspace into an expandable access port 100 that provides an elongated workspace 152.

FIGS. 33A and 33B illustrate the differential expansion of the primary arms 142 and secondary arms 134, which changes the arrangement of the distal primary arm ends 142b and distal secondary arm ends 144b from a circular arrangement to an elongated arrangement to form the elongated workspace 152.

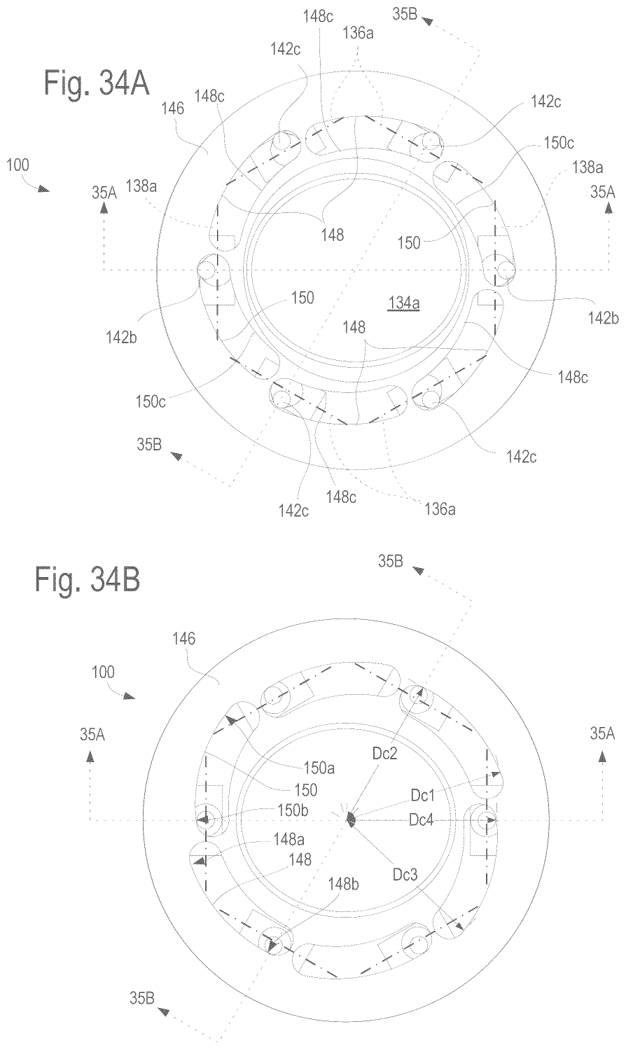

FIGS. 34A-35B illustrate another exemplary embodiment of an expandable access port 100 having an elongated membrane cross-section and an elongated workspace 150. In this case, the size of the elongated workspace 150 is provided by selecting different shapes for the primary cam surfaces 148 and the secondary cam surfaces 150. Each primary cam surface 148 extends from a first cam end 148a located at a first cam distance Dc1 from the longitudinal axis 140, to a second cam end 148b located at a second cam distance Dc2 from the longitudinal axis 140. Each secondary cam surface 150 extends from a first cam end 150a located at a third cam distance Dc3 from the longitudinal axis 140, to a second cam end 150b located at a fourth cam distance Dc4 from the longitudinal axis 140.

The first cam distance Dc1 and second cam distance Dc2, are selected to cause the primary arm distal ends 142b to move from a first distance D1 to a second distance D2 from the longitudinal axis 140. The third cam distance Dc3 and fourth cam distance Dc4, are selected to cause the secondary arm distal ends 144b to move to from a third distance D3 to a fourth distance D4 from the longitudinal axis 140. As with the previous embodiments (starting at FIG. 27A), the fourth distance D4 is greater than the second distance D2, and the third distance D3 is less than the fourth distance D4. (Distances D1, D2, D3 and D4 may be measured at an innermost edge of each arm 142, 144.)

The foregoing movement of the primary arm distal ends 142b and secondary arm distal ends 144b is achieved by making the difference in value between the first cam distance Dc1 and second cam distance Dc2 less than the difference in value between the third cam distance Dc3 and fourth cam distance Dc4 (i.e., Dc2−Dc1<Dc4−Dc3). Accordingly, the secondary arms 144 are displaced a greater distance than the primary arms 142 as the activation ring 146 is rotated from the first activation ring position to the second activation ring position.

The embodiment of FIGS. 34A-35B provides a benefit in that the arm mounting body 134 can be made with the first pivot location distances Dp1 equal to the second pivot location distances Dp2. Thus, the arm mounting body 134 can be used to make access ports providing circular workspaces and elongated workspaces. However, when the first pivot location distances Dp1 are equal to the second pivot location distances Dp2, the expandable membrane 118 can assume a non-elongated shape, either along its entire length or at the proximal membrane end 118a (see, e.g., FIG. 33A). This might cause undesirable excessive expansion of the brain tissue along the minor membrane axis Am2 as the device is inserted into the tissue. To avoid this, primary arms 142 may be constructed with an inward bend 142d towards the longitudinal axis 140, at which the expandable membrane 118 defines an elongated membrane axis, even when the device is in the closed position. More specifically, when the activation ring 146 is in the first activation ring position (i.e., the closed device position), the expandable membrane 118 may have an elongated cross-section at a proximal membrane position 118g located near, but not at, the arm mounting body 134. Similar arrangements can be used in other embodiments to modify the size of the elongated cross-sectional shape along the length of the arms.

As will be apparent from the foregoing, embodiments may be configured to provide an elongated membrane cross-section using various different arrangements of parts. In the embodiments of FIGS. 27A-31B, the pivot location distances Dp1, Dp2 are different, so as to create an elongated membrane cross-section beginning at a proximal membrane position located at the arm mounting body 134. In the embodiments of FIGS. 32A-35B, the pivot location distances Dp1, Dp2 are equal, so that the membrane cross section is not elongated at the arm mounting body 134, but can become elongated upon moving the device to the opened position. Also, in FIGS. 34A-35B, the arms may be shaped to create an elongated membrane cross-section located at a proximal membrane position that is spaced from the arm mounting body 134. Features of one embodiment may be used with features of other embodiments, as desired.

Embodiments of expandable access ports 100 providing an elongated membrane cross-section and an elongated workspace 152 can include additional features to provide one or more benefits. For example, each primary cam surface 148 and secondary cam surface 150 may have a counter-surface 148c, 150c (see FIG. 27A) that applies a closing force against the respective primary cam follower 142c or secondary cam follower 144c. By such interaction, the activation ring 146 can drive the primary arms 142 and secondary arms 144 towards the closed position by rotating the activation ring 146 from the second activation ring position to the first activation ring position.

Embodiments also may include any suitable number of primary arms 142 and secondary arms 144. Preferably, there are at least two primary arms 142 and at least two secondary arms 144. In the embodiments of FIGS. 27A-35B, there are four primary arms 142 and two secondary arms 144. In the embodiment of FIG. 36, there are two primary arms 142, and four secondary arms 144. In either case, each primary arm 142 may be arranged diametrically opposite another primary arm 142, and each secondary arm 144 may be arranged diametrically opposite another secondary arm 144.

Other embodiments may include additional arms that expand to a distance somewhere between the second distance D2 and the fourth distance D4. For example, FIGS. 37A and 37B show primary arms 142 that expand to a first distance D2 from the longitudinal axis 14, secondary arms 144 that expand to a fourth distance D4 from the longitudinal axis 140, and tertiary arms 154 that expand to a fifth distance D5 from the longitudinal axis 140. In this case, the fourth distance D4 is greater than the second distance D2 and the fifth distance D5 is greater than the second distance D2 and less than the fourth distance D4.

In still other embodiments, such as shown in FIG. 38, the primary arms 142 and secondary arms 144 may be arranged to create a workspace that is elongated along multiple different directions. This can be achieved by locating each secondary arm 144 opposite a primary arm 142, or by other arrangements.

In all of the embodiments, the primary arms 142 and secondary arms 144 may be equally distributed around the longitudinal axis 140 (e.g., at 60 degree increments for embodiments with a total of six arms 142, 144), or the arms 142, 144 may be distributed at unequal spacings.

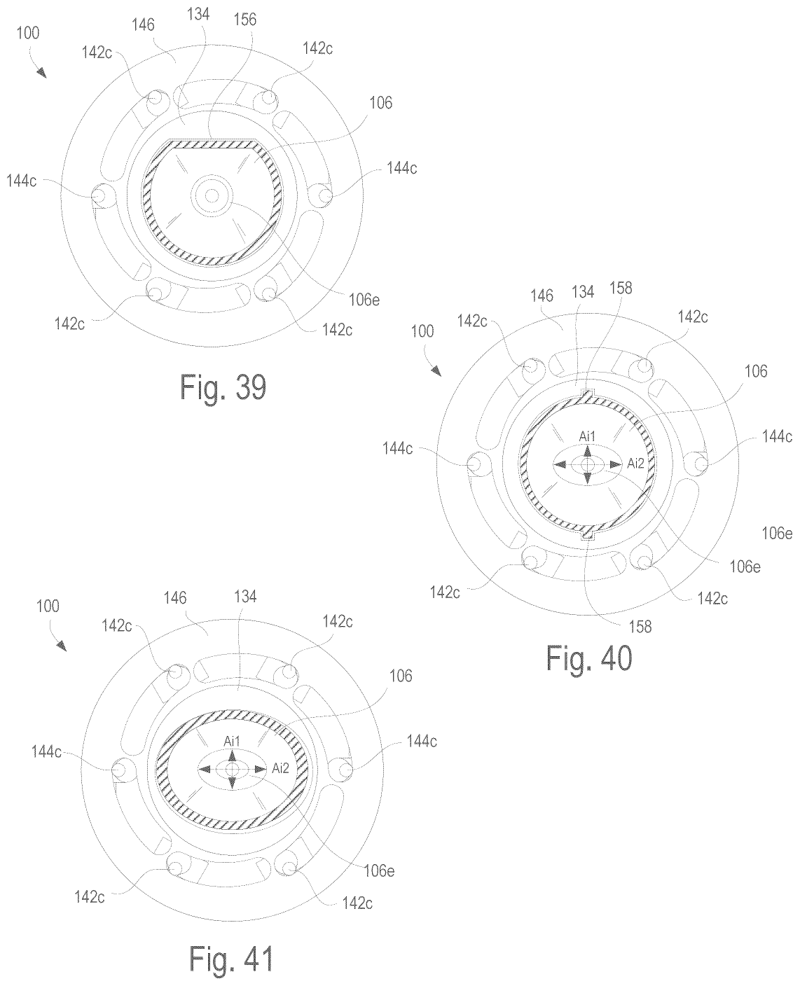

Referring now to FIGS. 39-41, expandable access ports 100 that provide an elongated workspace 152 can be used in conjunction with an introducer 106, such as described above. The introducer 106 may be hollow and configured to receive a navigation probe or other instruments. The introducer also may be solid. The introducer 106 may be constructed to selectively mount in a mounting position within the arm mounting body opening 134a with a proximal end of the introducer adjacent to the arm mounting body 134, and a distal introducer end extending beyond each primary arm distal end 142b and each secondary arm distal end 144b, in a manner comparable to the arrangement shown in FIGS. 1 and 12-14.

The proximal end of the introducer 106 may include a rotation indexing structure that engages a corresponding indexing structure on the arm mounting body 134, so as to only permit the introducer 106 and arm mounting body 134 to be assembled into the mounting position when the rotation indexing structures are properly aligned. For example, in FIG. 39, the introducer 106 and arm mounting body 134 have corresponding flats 156 that must be aligned to fully assemble the introducer 106 and the arm mounting body 134. In this case, the introducer 106 and arm mounting body 134 can be assembled in only one rotational position. In FIG. 40, the introducer 106 and arm mounting body 134 have corresponding ribs and slots 158 that allow the introducer 106 to be assembled with the arm mounting body 134 in two positions at 180 degrees from each other. Similarly, in FIG. 41, the introducer 106 has an oval (or other elongated) cross-section that fits into a correspondingly shaped arm mounting body opening 134a to allow the introducer 106 to be assembled with the arm mounting body 134 in two 180 degree opposite positions. In this case, the proximal introducer end is elongated and dimensioned to fit within the arm mounting body opening 134a without being rotatable about the longitudinal axis 140 relative to the arm mounting body 134.

As shown in FIG. 39, the introducer 106 can terminate at a circular tip 106e. This is preferred when the primary arms 142 and secondary arms 144 assume a circular configuration when in the closed position (e.g., as in FIG. 33A).

As shown in FIGS. 40 and 41, the introducer 106 can terminate at an elongated (e.g., oval) tip 106e having a major introducer axis dimension Ai1 that is larger than a minor introducer axis dimension Ai2 extending perpendicular to the major introducer axis dimension Ai1. This is preferred when the primary arms 142 and secondary arms 144 assume an elongated configuration when in the closed position (e.g., as in FIG. 29A). In this case, the introducer 106 and arm mounting body 134 may include rotation indexing structures (such as those described above or having other constructions) to assure that a primary introducer axis is aligned with the primary axis of the elongated workspace 152 when the parts are fully assembled.

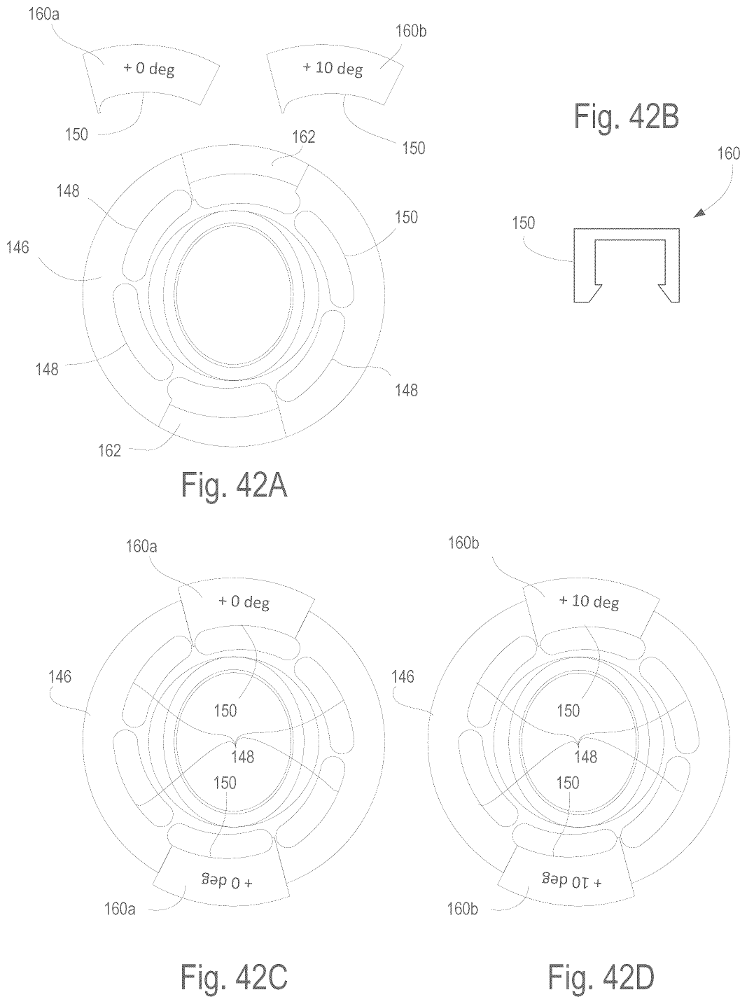

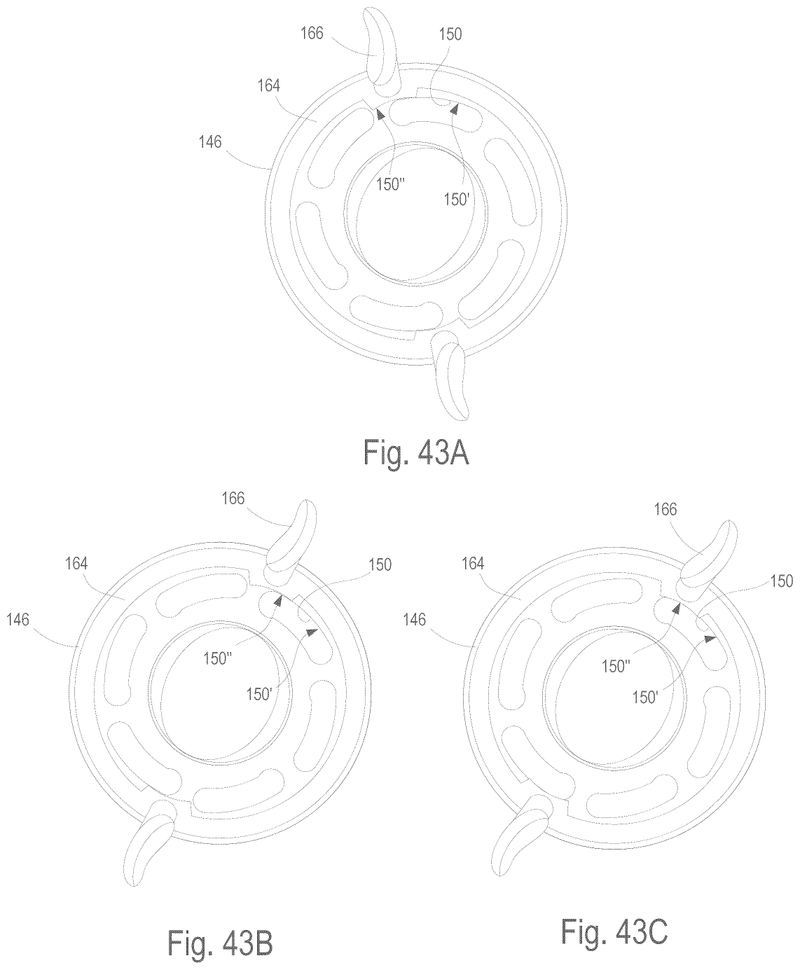

Referring now to FIGS. 42A-43C, some embodiments may be configured to selectively operate with or without creating an elongated workspace 152, or may be configured to selectively change the size (e.g., the dimension of the primary axis W1) of the elongated workspace 152.

In the embodiment of FIGS. 42A-42D the activation ring 146 includes one or more interchangeable cam surfaces. In this example, the primary cam surfaces 148 are integrally formed with the remainder of the activation ring 146, while the secondary cam surfaces 150 are formed on inserts 160. The inserts 160 may be installed at corresponding locations on the activation ring 146, to place the secondary cam surfaces 150 at the correct location to engage the secondary arms 144 (not shown). The inserts 160 may, for example, comprise clip-shaped parts that attach at corresponding recesses 162 of the activation ring 146. As shown in FIG. 42B, the inserts 160 may have hooked ends that hold the insert 160 via snap engagement to the activation ring 146. In other cases, the inserts 160 may be secured to the activation ring 146 permanently such as by adhesive or melt fusion, or affixed by a releasable fastener such as a screw.

The inserts 160 may be provided with various different secondary cam surfaces 150 having different arrangements of one or both of the third cam distance Dc3 and the fourth cam distance Dc4. By selecting different cam distances Dc3, Dc4, the rotation angle of the secondary arm 144 can be modified, to thereby change the value of the third distance D3 and/or the fourth distance D4. For example, in the embodiment of FIG. 42C, the insert 160a may have a secondary cam surface 150 with the third cam distance Dc3 and fourth cam distance Dc4 selected to rotate the secondary arms 144 a first angle to achieve a first value of the fourth distance D4 for a given length of the secondary arm 144. In contrast, the insert 160b of FIG. 42D might have a secondary cam surface 150 with the third cam distance Dc3 and fourth cam distance Dc4 selected to rotate the secondary arms 144 by a second angle (e.g., 10 degrees greater than the first angle) to achieve a larger value of the fourth distance D4 for the secondary arm 144.

Such selectable inserts 160 also may be used to provide a uniform fourth distance D4 for secondary arms 144 having different lengths. For example, an insert 160a providing a relatively small amount of rotation might be used with a longer secondary arm 144 to achieve a certain fourth distance D4 (e.g., 10 millimeters), while an insert 160b providing a relatively large amount of rotation might be used with a shorter secondary arm 144 to achieve the same fourth distance D4 (i.e., 10 millimeters).

In the foregoing embodiments, the elongated workspace 152 is created in one movement of the activation rings 154. This provides a simple and intuitive operation, and can minimize the complexity of the device. It also operates quickly to avoid unnecessarily prolonging the surgical procedure, which is particularly beneficial when used for surgery in the delicate tissue of the brain. However, other embodiments may allow selective operation of the secondary arms, as described, for example, in relation to FIGS. 43A-43C.

Another embodiment providing a variable displacement of the secondary arms 144 is shown in FIGS. 43A-43C. In this case, the activation ring 146 has primary cam surfaces 148 for driving the primary arms 142, and secondary cam surfaces 150 for driving the secondary arms 144. The secondary cam surfaces 150 include two segments 150′, 150″. The first segment 150′ is fixed relative to the activation ring 146, and the second segment 150″ is rotatable about the longitudinal axis 140 relative to the remainder of the activation ring 146. For example, the second segments 150″ may be mounted on a sub-ring 164 that is rotatably mounted to the remainder of the activation ring 146. Handles 166 may be provided on the sub-ring 164 to facilitate operation.

The first segment 150′ extends from a third cam distance Dc3 to a fourth cam distance Dc4, and the second segment 150″ extends from a first end that blends smoothly with a portion of the first segment 150′ (e.g. at a distance equal to Dc3, Dc4, or some value in between) to a second end located at a fifth cam distance Dc5 from the longitudinal axis 140. The fifth cam distance Dc5 is less than the fourth cam distance Dc4. The fourth cam distance Dc4 may be equal to or less than the third cam distance Dc3.

In use, the device starts in the fully closed position, as shown in FIG. 43A. The surgeon can rotate the activation ring 146 to drive the primary arms 142 to their opened positions, and move the secondary arms 144 partially to their opened positions (or maintain the secondary arms 144 in place), as shown in FIG. 43B. Then, the surgeon can rotate the second segments 150″ of the secondary cam surface 150 by rotating the sub-ring 164 to the position shown in FIG. 43C, to thereby move the secondary arms 144 to their fully-opened positions. In other embodiments, the order of operation can be reversed (e.g., the sub-ring 164 may be rotated, and then the remainder of the activation ring is 146 rotated). This embodiment allows rapid changes to the shape of the workspace 152 as desired during surgery.

The second segments 150″ may be provided to increase the elongation of the elongated workspace 152. For example, the primary cam surfaces 148 and the first segments 150′ of the secondary cam surfaces 150 may operate to create an elongated workspace 152 as described above, and then the second segments 150″ may be engaged to further expand the workspace 152. Alternatively, the second segments 150″ may be used to convert a non-elongated workspace into an elongated workspace 152. In this example, the third cam distance Dc3 may equal the first cam distance Dc1, and the fourth cam distance Dc4 may equal the second cam distance, so that the primary cam surfaces 148 and the first segments 150′ of the secondary cam surfaces 150 are the same shape and operate to move their respective arms 142, 144 by the same distance. The second segments 150″ of the secondary cam surfaces 150 terminate at the fifth cam distance Dc5 to move the secondary arms 144 further outward to the fourth distance D4, when it is desired to do so.

Other embodiments may include features to provide variable displacement of one or more primary arms 142. For example, the embodiment of FIGS. 42A-42D may be modified to accept inserts defining both the primary cam surfaces 148 and the secondary cam surfaces 150. As another example, the embodiment of FIGS. 43A-43C may be modified to selectively enlarge dimension D2 of the primary arms 142 to convert an elongated workspace 152 into a non-elongated workspace during the course of an operation. In other cases, only the primary cam surfaces 148 may be adjustable via inserts or other means.

The foregoing embodiments provide multiple different arrangements to provide an elongated workspace 152. Although discrete examples are shown, it will be readily understood by the person of ordinary skill in the art, in view of the disclosures herein, that embodiments may be modified or combined to provide other embodiments of expandable access ports having an elongated workspace.

Terms in the claims and specification identifying elements or features in the singular (e.g., “a” “an”) are not intended to be interpreted to require exclusively a single element or feature, unless explicitly stated or otherwise necessary or clear from the context. Similarly, in the claims and specification, the term “a number” is used with the meaning “one or more.”

The present disclosure provides a number of exemplary embodiments of the invention defined by the appended claims. The description of such embodiments is not intended to limit the scope of the claims beyond what is defined in the claims. It will also be understood that, while embodiments may provide particular advantages in certain cases, the scope of the claims is not limited to embodiments providing any particular advantage or functionality. It will further be appreciated that other embodiments encompassed by the claims may diverge from those described herein in both appearance and functionality, and the various features of particular embodiments described herein may be used with other embodiments without departing from the scope of the claims.

1. An expandable surgical access port comprising:
an arm mounting body defining an arm mounting body opening surrounding a longitudinal axis, wherein the arm mounting body comprises:
a number of primary pivot locations surrounding the longitudinal axis and fixed at a respective first pivot location distance from the longitudinal axis, and
a number of secondary pivot locations surrounding the longitudinal axis and fixed at a respective second pivot location distance from the longitudinal axis;
a number of primary arms arranged around the longitudinal axis, wherein each primary arm is pivotally attached at a respective primary pivot location and extends in a distal direction along the longitudinal axis to a respective primary arm distal end;
a number of secondary arms arranged around the longitudinal axis, wherein each secondary arm is pivotally attached at a respective secondary pivot location and extends in the distal direction along the longitudinal axis to a respective secondary arm distal end;
an activation ring rotatably mounted to the arm mounting body to rotate about the longitudinal axis between a first activation ring position and a second activation ring position, the activation ring comprising:
a number of primary cam surfaces configured to each engage a respective primary arm to drive the respective primary arm from a respective closed position in which the respective primary arm distal end is a respective first arm distance from the longitudinal axis to a respective open position in which the respective primary arm distal end is a respective second arm distance from the longitudinal axis, upon movement of the activation ring from the first activation ring position to the second activation ring position, and
a number of secondary cam surfaces configured to each engage a respective secondary arm to drive the respective secondary arm from a respective closed position in which the respective secondary arm distal end is a respective third arm distance from the longitudinal axis to a respective open position in which the respective secondary arm distal end is a respective fourth arm distance from the longitudinal axis, upon movement of the activation ring from the first activation ring position to the second activation ring position;
wherein the respective fourth arm distance of each secondary arm distal end is greater than the respective second arm distance of each primary arm distal end; and
an expandable membrane extending from a proximal membrane position, to a distal membrane position adjacent to each primary arm distal end and each secondary arm distal end;
wherein, when the activation ring is in at least the second activation ring position, an entirety of the expandable membrane between the proximal membrane position and the distal membrane position comprises an elongated membrane cross-section as viewed along the longitudinal axis, the elongated membrane cross-section extending along a major membrane axis and a minor membrane axis perpendicular to the major membrane axis.

2. The expandable surgical access port of claim 1, the entirety of the expandable membrane between the proximal membrane position and the distal membrane position comprises the elongated membrane cross-section when the activation ring is in the first activation ring position.

3. The expandable surgical access port of claim 1, wherein the proximal membrane position is located at the arm mounting body.

4. The expandable surgical access port of claim 1, wherein the respective second pivot location distance of each secondary pivot location is greater than the respective first pivot location distance of each primary pivot location.

5. The expandable surgical access port of claim 1, wherein the arm mounting body opening comprises an elongated opening cross-section as viewed along the longitudinal axis, extending along a major opening axis and a minor opening axis perpendicular to the major opening axis, and the major opening axis is aligned with the major membrane axis.

6. The expandable surgical access port of claim 1, wherein:
each primary pivot location defines a respective primary pivot axis extending in a plane perpendicular to the longitudinal axis and tangential to the longitudinal axis; and
each secondary pivot location defines a respective secondary pivot axis extending in a plane perpendicular to the longitudinal axis and tangential to the longitudinal axis.

7. The expandable surgical access port of claim 1, wherein:
each primary cam surface extends about the longitudinal axis from a respective first cam end at a first cam distance from the longitudinal axis to a respective second cam end at a second cam distance from the longitudinal axis, wherein the second cam distance is less than the first cam distance; and
each secondary cam surface extends about the longitudinal axis from a respective first cam end at a third cam distance from the longitudinal axis to a respective second cam end at a fourth cam distance from the longitudinal axis, wherein the fourth cam distance is less than the third cam distance;
wherein the first cam distance is equal to the third cam distance, and the second cam distance is equal to the fourth cam distance.

8. The expandable surgical access port of claim 1, wherein:
each primary cam surface extends about the longitudinal axis from a respective first cam end at a first cam distance from the longitudinal axis to a respective second cam end at a second cam distance from the longitudinal axis, wherein the second cam distance is less than the first cam distance; and
each secondary cam surface extends about the longitudinal axis from a respective first cam end at a third cam distance from the longitudinal axis to a respective second cam end at a fourth cam distance from the longitudinal axis, wherein the fourth cam distance is less than the third cam distance;
wherein the first cam distance is less than the third cam distance and the second cam distance is less than the fourth cam distance.

9. The expandable surgical access port of claim 1, wherein:
each primary arm comprises a primary cam follower located a first cam follower distance from a respective primary pivot axis of the respective primary arm pivot;
each secondary arm comprises a secondary cam follower located a second cam follower distance from a respective secondary pivot axis of the respective secondary arm pivot; and
the second cam follower distance is less than the first cam follower distance.

10. The expandable surgical access port of claim 1, wherein:
each primary cam surface extends about the longitudinal axis from a respective first cam end at a first cam distance from the longitudinal axis to a respective second cam end at a second cam distance from the longitudinal axis, wherein the second cam distance is less than the first cam distance; and
each secondary cam surface extends about the longitudinal axis from a respective first cam end at a third cam distance from the longitudinal axis to a respective second cam end at a fourth cam distance from the longitudinal axis, wherein the fourth cam distance is less than the third cam distance;
wherein a difference in value between the first cam distance and the second cam distance is less than a difference in value between the third cam distance and the fourth cam distance.

11. The expandable surgical access port of claim 1, wherein at each of the primary arms comprises an inward bend towards the longitudinal axis.

12. The expandable surgical access port of claim 11, wherein the respective second pivot location distance of each secondary pivot location is equal to the respective first pivot location distance of each primary pivot location.

13. The expandable surgical access port of claim 1, wherein the activation ring is secured to the arm mounting body to remain at a fixed location along the longitudinal axis, relative to the arm mounting body, throughout rotation of the activation ring between the first activation ring position and the second activation ring position.

14. The expandable surgical access port of claim 1, wherein the activation ring comprises a plurality of closure cam surface configured to each engage a respective primary arm or secondary arm such that, upon rotation of the activation ring from the second activation ring position to the first activation ring position, the plurality of closure cam surfaces:
generate a closing force to move each respective primary arm from the respective opened position to the respective closed position; and
generate a closing force to move each respective secondary arm from the respective opened position to the respective closed position.

15. The expandable surgical access port of claim 1, wherein:
the number of primary arms equals at least two;
the number of secondary arms equals at least two;
each primary arm is diametrically opposite another primary arm relative to the longitudinal axis; and
each secondary arm is diametrically opposite another secondary arm relative to the longitudinal axis.

16. The expandable surgical access port of claim 1, further comprising an introducer configured to selectively mount within the arm mounting body opening at a mounting position in which a proximal introducer end is adjacent to the arm mounting body and a distal introducer end extends in the distal direction beyond each primary arm distal end and each secondary arm distal end.

17. The expandable surgical access port of claim 16, wherein the proximal introducer end is elongated and dimensioned to fit within the arm mounting body opening without being rotatable about the longitudinal axis relative to the arm mounting body.

18. The expandable surgical access port of claim 17, wherein:
the distal introducer end comprises an elongated introducer cross-section as viewed along the longitudinal axis, with a major introducer axis and a minor introducer axis extending perpendicular to the major introducer axis; and
the major introducer axis is aligned with the major opening axis when the introducer is in mounted to the arm mounting body in the mounting position.

19. The expandable surgical access port of claim 1, wherein each secondary cam surface comprises a separate part that is selectively secured to the activation ring.

20. The expandable surgical access port of claim 1, wherein each secondary cam surface is rotatable about the longitudinal axis through a range of motion independently of each primary cam surface.
